SEQUENCE LISTING (53 sequences)

SEQ ID NO: 1 - protein, 365 aa, Flavobacterium sp.
MQTRRVVLKSAAAAGTLLGGLAGCASVAGSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 2 - protein, 336 aa, Artificial Sequence
  OPH
SIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 3 - protein, 384 aa, Agrobacterium radiobacter
MQTRRDALKSAAAITLLGGLAGCASMARPIGTGDLINTVRGPIPVSEAGFTLTHEHICGSSAGFLRAWPEFFGSRKALAEKAVRGLRHARSAGVQTIVDVSTFDIGRDVRLLAEVSRAADVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 4 - protein, 356 aa, Artificial Sequence
  OPDA
PIGTGDLINTVRGPIPVSEAGFTLTHEHICGSSAGFLRAWPEFFGSRKALAEKAVRGLRHARSAGVQTIVDVSTFDIGRDVRLLAEVSRAADVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 5 - protein, 292 aa, Escherichia coli
MSFDPTGYTLAHEHLHIDLSGFKNNVDCRLDQYAFICQEMNDLMTRGVRNVIEMTNRYMGRNAQFMLDVMRETGINVVACTGYYQDAFFPEHVATRSVQELAQEMVDEIEQGIDGTELKAGIIAEIGTSEGKITPLEEKVFIAAALAHNQTGRPISTHTSFSTMGLEQLALLQAHGVDLSRVTVGHCDLKDNLDNILKMIDLGAYVQFDTIGKNSYYPDEKRIAMLHALRDRGLLNRVMLSMDITRRSHLKANGGYGYDYLLTTFIPQLRQSGFSQADVDVMLRENPSQFFQ

SEQ ID NO: 6 - protein, 326 aa, Mycobacterium tuberculosis
MPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 7 - protein, 365 aa, Chryseobacterium balustinum
MQTRRVVLKSAAAAGTLLGRLAGCASVAGSIGTGDRINTVRGPITNSEAGFTLTHEHICGTSAGFLRAWQEFFGSRKALAEKAVRGLRRARAAGVRTIVDVSTFDIGRDVSLLAEVSMMVDVSLLAETGLWFDPPLSIGLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 8 - protein, 314 aa, Sulfolobus solfataricus
MRIPLVGKDSIESKDIGFTLIHEHLRVFSEAARQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 9 - protein, 314 aa, Artificial Sequence
  mutant phosphotriesterase
MRIPLVGKDSIESKDIGFTLIHEHLRVFSEAARQQWPHLYNEDEEFRNAVNEVKRAMQFGVKTIVDPTVMGLGRDIRFMEKVVKATGINLVAGTGIWIFIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDHYGLDLFLPVDKRNETTLRLIKDGYSDKIMISHDYLCTIDWGTAKPEYKPKLAPRWSITLIFEDTIPFLKRNGVNEEVIATIFKENPKKFFS

SEQ ID NO: 10 - protein, 314 aa, Artificial Sequence
  mutant phosphotriesterase
MRIPLVGKDSIESKDIGFTLIHEHLRAFSEAARQQWPHLYNEDEEFRNAVNEVKRAMQFGVKTIVDPTVMGIGRDIRFMEKVVKATGINLVAGTGIWIFIDLPFYFLNRSIDEIADLFIHDIKEGIQGTLNKAGFVKIAATEPGITKDVEKVIRAAAIANKETKVPIITHSNAHNNTGLEQQRILTEEGVDPGKILIGHLGDTDNIDYIKKIADKGSFIGLDHYPHDLFLPVDKRNETTLRLIKDGYSDKIMISHDYLCTIDWGTAKPEYKPKLAPRWSITLIFEDTIPFLKRNGVNEEVIATIFKENPKKFFS

SEQ ID NO: 11 - protein, 314 aa, Artificial Sequence
  mutant phosphotriesterase
MRIPLVGKDSIESKDIGFTLIHEHLRAFSEAARQQWPHLYNEDEEFRNAVNEVKRAMQFGVKTIVDVSVMGIGRDIRFMEKVVKATGINLVAGTGIWIFIDLPFYFLNRSIDEIADLFIHDIKEGIQGTLNKAGFVKIAATEPGITKDVEKVIRAAAIANKETKVPIITHSNAHNNTGLEQQRILTEEGVDPGKILIGHLGDTDNIDYIKKIADKGSFIGLDHYPHDLSLPVDKRNETTLRLIKDGYSDKIMISHDYLCTIDAGTAKPEYKPKLAPRISITLIFEDTIPFLKRNGVNEEVIATIFKENPKKFFS

SEQ ID NO: 12 - protein, 315 aa, Sulfolobus acidocaldarius
MTKIPLVGKGEISPGEMGFTLIHEHLRVFSEPVRYQWPHLYNEDEELKNAVNEVKTIMSYGVKTIVDPTVMGLGRDIRFSEKVVKETGINVIAATGLYTYTDLPFFFNGRSLEEIAELLIHDIKKGIQGTNNRAGFIKVAADEPGITRDVERAIRAAAIAQKETNVPIITHSNAHNGTGLEQQRILMEEGVDPGRVLIGHLGDTDNVDYIKKIADKGSFVGLDRYGLDLFLPIDKRNEVLLKLIKDGYLDRIMVSQDYCCTIDWGIAKPEYKPKLAPKWSMSLIFTDVIPSIKRAGVTDEQLHVIFVKNPARLFS

SEQ ID NO: 13 - protein, 315 aa, Artificial Sequence
  mutant phosphotriesterase
MTKIPLVGKGEISPGEMGFTLIHEHLRVFSEPVRYQWPHLYNEDEELKNAVNEVKTIMSYGVKTIVDPTVMGLGRDIRFSEKVVKETGINVIAATGLWTFTDLPFFFNGRSLEEIAELLIHDIKKGIQGTNNRAGFIKVAADEPGITRDVERAIRAAAIAQKETNVPIITHSNAHNGTGLEQQRILMEEGVDPGRVLIGHLGDTDNVDYIKKIADKGSFVGLDHYGLDLFLPIDKRNEVLLKLIKDGYLDRIMVSQDYLCTIDWGIAKPEYKPKLAPKWSMSLIFTDVIPSIKRAGVTDEQLHVIFVKNPARLFS

SEQ ID NO: 14 - protein, 315 aa, Artificial Sequence
  mutant phosphotriesterase
MTKIPLVGKGEISPGEMGFTLIHEHLRAFSEPVRYQWPHLYNEDEELKNAVNEVKTIMSYGVKTIVDPTVMGIGRDIRFSEKVVKETGINVIAATGLWTFTDLPFFFNGRSLEEIAELLIHDIKKGIQGTNNRAGFIKVAATEPGITRDVERAIRAAAIAQKETNVPIITHSNAHNGTGLEQQRILMEEGVDPGRVLIGHLGDTDNVDYIKKIADKGSFVGLDHYPHDLFLPIDKRNEVLLKLIKDGYLDRIMVSQDYLCTIDWGIAKPEYKPKLAPKWSMSLIFTDVIPSIKRAGVTDEQLHVIFVKNPARLFS

SEQ ID NO: 15 - protein, 315 aa, Artificial Sequence
  mutant phosphotriesterase
MTKIPLVGKGEISPGEMGFTLIHEHLRAFSEPVRYQWPHLYNEDEELKNAVNEVKTIMSYGVKTIVDVSVMGIGRDIRFSEKVVKETGINVIAATGLWTFTDLPFFFNGRSLEEIAELLIHDIKKGIQGTNNRAGFIKVAATEPGITRDVERAIRAAAIAQKETNVPIITHSNAHNGTGLEQQRILMEEGVDPGRVLIGHLGDTDNVDYIKKIADKGSFVGLDHYPHDLSLPIDKRNEVLLKLIKDGYLDRIMVSQDYLCTIDAGIAKPEYKPKLAPKISMSLIFTDVIPSIKRAGVTDEQLHVIFVKNPARLFS

SEQ ID NO: 16 - protein, 355 aa, Homo sapiens
MAKLIALTLLGMGLALFRNHQSSYQTRLNALREVQPVELPNCNLVKGIETGSEDMEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 17 - protein, 351 aa, Musca domestica
MPIVQTVLGPIDPNLLGRTLTHEHIALDFEGFHCDPPEDFKPFLEGKLRMENLGFVRQYPYSSRDNTRFYDEETRVAVLKDIELYKKFGGGTIVENSSHGLKRNMEFMVEASKKTGVHIVAGTGHYVHDLQEKDHVGMTVEQMTDLYSKEIITGVELEGVGMVKCGFIGEVGSVYPIHDFEKRAIKATGEIQEVLGCGVSFHPGRDAEAPFEIVRLYLEAGGKASKCVMSHLDRTIFDIDQLLEFAKMGTYIQYDLFGTECSLYQLNLTVDMPSDAQRINNLMRLIDEGYVDKILMSHDLHTKHRLTSFGGHGYHHVHMNILPRMFAKGLSVEQIEQMTVTNPAKWLEFNI

SEQ ID NO: 18 - protein, 350 aa, Drosophila melanogaster
MSTVQTVLGTITPNLLGRTLTHEHVALDFEHFYRPPPPDFESELKAKISMSTLGYVRLYPYSSKENVRFYDGEALEAAKKDVLLYKKHGGGSIVENSSYGLKRNLEFIVELAKSTGVHFIAGTGHYIHAMQDASHASLTVEQMSDLYSKDIITGLQVNGKVVKCGFIGEVASVYPIHDFEKNAIKAAGEIQEVLGCGVSMHPHRVTKAPFEIMRLYLEAGGRADKCVMSHLDRTIFDIDELLEFAKLGCYIQYDLFGTECSFYQLNTSVDMISDGQRIDNLIKLIKEGLVDKLLMSHDIHTKHRLTSYGGHGYHHIHTNILPRMFDRGVTLEQVEQMTVTNPAKWLAFDP

SEQ ID NO: 19 - protein, 384 aa, Artificial Sequence
  mutant OPDA
MQTRRDALKSAAAITLLGGLAGCASMARPIGTGDLINTVRGSIPVSEAGFTLTHEHICGSSAGFLRAWPEFFGSRKALAEKAVRGLRHARSAGVQTIVDVSTFDIGRDVRLLAEVSRAADVHIVAATGLWFDPSLSMRMRSVEELTQFFLREIQHGIEDTGIRAGIIKVSTTGKATPFQELVLKAAARASLATGVPVTTHTSASQRDGEQQAAIFESEGLSPSRVCIGHSDDTDDLGYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 20 - protein, 356 aa, Artificial Sequence
  mutant OPDA
PIGTGDLINTVRGSIPVSEAGFTLTHEHICGSSAGFLRAWPEFFGSRKALAEKAVRGLRHARSAGVQTIVDVSTFDIGRDVRLLAEVSRAADVHIVAATGLWFDPSLSMRMRSVEELTQFFLREIQHGIEDTGIRAGIIKVSTTGKATPFQELVLKAAARASLATGVPVTTHTSASQRDGEQQAAIFESEGLSPSRVCIGHSDDTDDLGYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 21 - protein, 384 aa, Artificial Sequence
  mutant OPDA
MQTRRDALKSAAAITLLGGLAGCASMARPIGTGDLINTVRGPIPVSEAGFTLTHEHICGSSAGFLRAWPEFFGSRKALAEKAVRGLRHARSAGVQTIVDVSTFDIGRDVRLLAEVSRADDVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 22 - protein, 356 aa, Artificial Sequence
  mutant OPDA
PIGTGDLINTVRGPIPVSEAGFTLTHEHICGSSAGFLRAWPEFFGSRKALAEKAVRGLRHARSAGVQTIVDVSTFDIGRDVRLLAEVSRADDVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 23 - protein, 384 aa, Artificial Sequence
  mutant OPDA
MQTRRDALKSAAAITLLGGLAGCASMARPIGTGDLINTVRGPIPVSEAGFTLTHEHICGSSAGFLRAWPEFFGSRKALAEKAVRGLRHARSAGVQTIVDVSTFDIGRDVRLLAEVSRAHDVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSFVTNIMDVMDRINPDGMAFVPLRVIPFLREKGVPPETLAGVTVANPARFLSPTVRAVVTRSETSRPAAPIPRQDTER

SEQ ID NO: 24 - protein, 356 aa, Artificial Sequence
  mutant OPDA
PIGTGDLINTVRGPIPVSEAGFTLTHEHICGSSAGFLRAWPEFFGSRKALAEKAVRGLRHARSAGVQTIVDVSTFDIGRDVRLLAEVSRAHDVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSFVTNIMDVMDRINPDGMAFVPLRVIPFLREKGVPPETLAGVTVANPARFLSPTVRAVVTRSETSRPAAPIPRQDTER

SEQ ID NO: 25 - protein, 30 aa, Artificial Sequence
  peptide signal
MKKRRVVNSVLLLLLLASALALTVAPMAKA

SEQ ID NO: 26 - protein, 46 aa, Artificial Sequence
  peptide signal
MEARMTGRRKVTRRDAMADAARAVGVACLGGFSLAALVRTASPVDA

SEQ ID NO: 27 - protein, 41 aa, Artificial Sequence
  peptide signal
MSRSAKPQNGRRRFLRDVVRTAGGLAAVGVALGLQQQTARA

SEQ ID NO: 28 - protein, 27 aa, Artificial Sequence
  peptide signal
MTWSRRQFLTGVGVLAAVSGTAGRVVA

SEQ ID NO: 29 - protein, 27 aa, Artificial Sequence
  peptide signal
MDRRRFLTLLGSAGLTATVATAGTAKA

SEQ ID NO: 30 - protein, 37 aa, Artificial Sequence
  peptide signal
MSEKDKMITRRDALRNIAVVVGSVATTTMMGVGVADA

SEQ ID NO: 31 - protein, 34 aa, Artificial Sequence
  peptide signal
MQIVNLTRRGFLKAACVVTGGALISIRMTGKAVA

SEQ ID NO: 32 - protein, 45 aa, Artificial Sequence
  peptide signal
MNNEETFYQAMRRQGVTRRSFLKYCSLAATSLGLGAGMAPKIAWA

SEQ ID NO: 33 - protein, 48 aa, Artificial Sequence
  peptide signal
MSTGTTNLVRTLDSMDFLKMDRRTFMKAVSALGATAFLGTYQTEIVNA

SEQ ID NO: 34 - protein, 50 aa, Artificial Sequence
  peptide signal
MKCYIGRGKNQVEERLERRGVSRRDFMKFCTAVAVAMGMGPAFAPKVAEA

SEQ ID NO: 35 - protein, 26 aa, Artificial Sequence
  peptide signal
MNRRNFIKAASCGALLTGALPSVSHA

SEQ ID NO: 36 - protein, 44 aa, Artificial Sequence
  peptide signal
MSHADEHAGDHGATRRDFLYYATAGAGTVAAGAAAWTLVNQMNP

SEQ ID NO: 37 - protein, 44 aa, Artificial Sequence
  peptide signal
MTQISGSPDVPDLGRRQFMNLLTFGTITGVAAGALYPAVKYLIP

SEQ ID NO: 38 - protein, 32 aa, Artificial Sequence
  peptide signal
MDRRTFLRLYLLVGAAIAVAPVIKPALDYVGY

SEQ ID NO: 39 - protein, 42 aa, Artificial Sequence
  peptide signal
MTKLSGQELHAELSRRAFLSYTAAVGALGLCGTSLLAQGARA

SEQ ID NO: 40 - protein, 31 aa, Artificial Sequence
  peptide signal
MTLTRREFIKHSGIAAGALVVTSAAPLPAWA

SEQ ID NO: 41 - protein, 31 aa, Artificial Sequence
  peptide signal
MTISRRDLLKAQAAGIAAMAANIPLSSQAPA

SEQ ID NO: 42 - protein, 32 aa, Artificial Sequence
  peptide signal
MSEALSGRGNDRRKFLKMSALAGVAGVSQAVG

SEQ ID NO: 43 - protein, 45 aa, Artificial Sequence
  peptide signal
MKTKIPDAVLAAEVSRRGLVKTTAIGGLAMASSALTLPFSRIAHA

SEQ ID NO: 44 - protein, 35 aa, Artificial Sequence
  peptide signal
MSNFNQISRRDFVKASSAGAALAVSNLTLPFNVMA

SEQ ID NO: 45 - protein, 30 aa, Artificial Sequence
  peptide signal
MSISRRSFLQGVGIGCSACALGAFPPGALA

SEQ ID NO: 46 - protein, 37 aa, Artificial Sequence
  peptide signal
MKTVLPSVPETVRLSRRGFLVQAGTITCSVAFGSVPA

SEQ ID NO: 47 - protein, 44 aa, Artificial Sequence
  peptide signal
MGRLNRFRLGKDGRREQASLSRRGFLVTSLGAGVMFGFARPSSA

SEQ ID NO: 48 - protein, 50 aa, Artificial Sequence
  peptide signal
MSDKDSKNTPQVPEKLGLSRRGFLGASAVTGAAVAATALGGAVMTRESWA

SEQ ID NO: 49 - protein, 32 aa, Artificial Sequence
  peptide signal
MESRTSRRTFVKGLAAAGVLGGLGLWRSPSWA

SEQ ID NO: 50 - protein, 27 aa, Artificial Sequence
  peptide signal
MSLSRRQFIQASGIALCAGAVPLKASA

SEQ ID NO: 51 - protein, 30 aa, Artificial Sequence
  peptide signal
MTLNRRDFIKTSGAAVAAVGILGFPHLAFG

SEQ ID NO: 52 - protein, 45 aa, Artificial Sequence
  peptide signal
MTDSRANRADATRGVASVSRRRFLAGAGLTAGAIALSSMSTSASA

SEQ ID NO: 53 - protein, 337 aa, Artificial Sequence
  OPH
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

(19) United States
(12) Patent Application Publication
(10) Pub. No.: US 2015/0337276 A1
(43) Pub. Date: Nov. 26, 2015

(54) ENZYME HAVING A NMDA RECEPTOR ANTAGONIST ACTIVITY AND/OR AN ANTICHOLINERGIC ACTIVITY

(71) Applicant: Olivier-Georges BOUMENDIL, Paris (FR)

(72) Inventor: Olivier-Georges BOUMENDIL, Paris (FR)

(21) Appl. No.: 14/654,780

(22) PCT Filed: Dec. 20, 2013
(86) PCT No.: PCT/EP2013/077780

§ 371 Date: Jun. 22, 2015

Related U.S. Application Data
(60) Provisional application No. 61/739,776, filed on Dec. 20, 2012.

(51) Int. Cl. C12N 9/16; A61L 31/16; A61L 29/16; A61L 31/08; A61K 38/46; A61L 29/08

(52) U.S. Cl. CPC C12N 9/16; A61K 38/465; A61L 29/08; A61L 29/16; A61L 31/08; A61L 31/16; C12Y 301/08001

(57) ABSTRACT

An enzyme having a NMDA antagonist activity and/or an anticholinergic activity, wherein the enzyme is selected from the group including phosphotriesterases and phosphotriesterases derivatives. A method for treating a disease, disorder or condition of the central nervous system in a subject, wherein the method includes administering to the subject the enzyme is also described.

### FIELD OF INVENTION

The present invention relates to the field of therapeutic treatment of diseases related to an over-activation of receptors of the central nervous system. More specifically, the present invention relates to enzymes having a NMDA receptor antagonist activity and/or an anticholinergic activity, and to the use of these enzymes for treating diseases related to the over-activation of the NMDA receptor and/or to the over-activation of the cholinergic receptors, such as, for example, neuropathic pain.

### BACKGROUND OF INVENTION

In Vertebrates, glutamate is the main excitatory neurotransmitter. N-methyl-D-aspartate (NMDA) receptors are one of the three types of ionotropic glutamate receptors in the central nervous system, playing critical roles in excitatory neurotransmission and synaptic plasticity, required for learning and memorization phenomena. The activity of NMDA receptors is negatively modulated by a variety of extracellular ions, such as, for example, Mg²⁺ and Zn²⁺, which can exert tonic inhibition under physiological conditions.

Over-activation of NMDA receptors leads to the continuous import of cations, especially of Ca²⁺ ions, within the post-synaptic neurons. This may cause neuropathologic clinical symptoms, such as, for example, excito-toxicity and postoperative hyperalgesia; and/or psychopathologic clinical symptoms, such as, for example, schizophrenia or opioid dependence.

Cholinergic receptors are also transmembrane proteins of the ionotropic receptors family present on the postsynaptic neurons membrane. Upon activation, presynaptic neurons secrete acetylcholine in the synaptic cleft, which will induce a signal upon fixation on cholinergic receptors of the postsynaptic neuron. In physiological states, acetylcholine is rapidly degraded in the synaptic cleft by an enzyme, acetylcholinesterase (AChE). Thus, inhibition of AChE favors the over-activation of cholinergic receptors. As for the NMDA receptor, the over-activation of cholinergic receptors may also lead to neuropathologic clinical symptoms, such as, for example, spasticity, and/or to psychopathologic clinical symptoms, such as, for example, schizophrenia or opioid dependence.

In order to treat diseases associated with neuropathologic clinical symptoms and/or to psychopathologic clinical symptoms, there is thus a need for compounds that may (i) inhibit NMDA receptors, and/or (ii) have an anticholinergic activity.

Phosphotriesterases (PTE) are metalloenzymes previously known to hydrolyze organophosphorus compounds (OP), and in particular phosphotriesters. The metal ion involved in the hydrolytic activity of PTE is a catalytic ion, which may be selected from Co²⁺, Fe²⁺, or Zn²⁺. OP are neurotoxic agents used as pesticides or as chemical weapons (OP are thus basic ingredients of sarin), causing behavioral problems, convulsions and brain lesions. Toxicity of OP may be due, at least in part to the definitive inactivation of AChE, but also to the deregulation of the glutamate metabolism with the over-activation of the NMDA receptor.

The inventors showed that PTE or PTE derivatives may inhibit NMDA receptors and exhibit an anticholinergic activity in absence of OP intoxication. These enzymes may thus be used for treating diseases, disorders or conditions related to the over-activation of the NMDA receptor and/or to the over-activation of cholinergic receptors, such as, for example, hyperalgesia, excito-toxicity, drug-related neurotoxicity, abnormal spinal and central spasticity, psychosis, stroke, Alzheimer' s disease, opioid dependence, blepharospasm, or hiccup. Without willing to be bound to a theory, the inventors suggest that these PTE or PTE derivatives enzymes act selectively in intrasynaptic, by chelating zinc and/or by acting on phosphate metabolism (such as, for example, on the metabolism of ATP and/or AMPc, which are known to be involved in synaptic function). In the post synaptic cell, these PTE or PTE derivatives enzymes act by inhibition of phosphate activity and Ca²⁺ dependant protein kinase II clusters.

### SUMMARY

The present invention thus relates to an enzyme having a NMDA antagonist activity and/or an anticholinergic activity, wherein said enzyme is selected from the group comprising phosphotriesterases and phosphotriesterases derivatives. In one embodiment of the invention, the enzyme has a phosphotriesterase activity. In another embodiment of the invention, the enzyme is a phosphotriesterase derivative and has a phosphomonoesterase activity. In another embodiment, the enzyme is a phosphotriesterase derivative and is not capable of hydrolyzing an organophosphorous molecule, preferably phosmet and/or fenthion.

The present invention also relates to a method for inhibiting a NMDA receptor, comprising administering an enzyme as hereinabove described, wherein said enzyme has a NMDA antagonist activity.

The present invention also relates to a method for inhibiting a cholinergic pathway, preferably for activating an acetylcholinesterase enzyme, comprising administering an enzyme according to the invention, wherein said enzyme has an anticholinergic activity.

Another object of the invention is a method for treating a disease, disorder or condition of the central nervous system in a subject in need thereof, wherein said method comprises administering to the subject an enzyme having a NMDA antagonist activity and/or an anticholinergic activity, wherein said enzyme is selected from the group comprising phosphotriesterases and phosphotriesterases derivatives.

In one embodiment, the disease, disorder or condition of the central nervous system is a NMDA related condition. In one embodiment, the disease, disorder or condition of the central nervous system is a NMDA related condition selected from the group comprising hyperalgesia, such as, for example, hyperalgesia induced by morphine treatment (such as, for example, during surgery, cancer treatment or in patients in final phase), hyperalgesia induced by opiod treatment (such as, for example, during orthopedic or digestive surgery, or in carcinology), neuropathies, such as, for example, neuropathic pain, intractable neuropathic pain, allodynia, pain wind up, excito-toxicity, such as, for example, traumatic excito-toxicity, vascular excito-toxicity, deafness related excito-toxicity or degenerative excito-toxicity, stroke and vascular conditions such as, for example, systemic vascularitis, Crohn disease, ulcerative colitis, collagenosis disease, Polyangeitis, necrotizing glomerulonephritis, Wegener granulomatosis, Polyarteritis nodosa, Giant cell arteritis (Horton disease), Kawasaki, Henoch-Schoenlein purpura, Cryoglobulinemia, Alzheimer's disease, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome and Childhood disintegrative disorder.

In another embodiment of the invention, the disease, disorder or condition of the central nervous system is an acetylcholine related condition. In one embodiment, the disease, disorder or condition of the central nervous system is an acetylcholine related condition, selected from the group comprising spasticity, Alzheimer' s disease, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome and Childhood disintegrative disorder.

In another embodiment of the invention, the disease, disorder or condition of the central nervous system is spinal or central spasticity induced by or related to a disease, disorder or condition selected from the list comprising spinal injury, post-traumatic spinal and cerebral sequels, multiple sclerosis or other demyelinating diseases (such as, for example, neuromyelitis and encephalomyelitis), myopathic syndrome, syringomyelia, encephalopathy (such as, for example, related to HIV), bladder instability and urination or micturition disorders with bladder spasticity.

In one embodiment of the invention, wherein the disease, disorder or condition of the central nervous system is an autonomous nervous system related condition. In one embodiment, the disease, disorder or condition of the central nervous system is an autonomous nervous system related condition selected from the group comprising blepharospasm, tinnitus and pathologic hiccup.

The present invention also relates to an enzyme having a NMDA antagonist activity, wherein said enzyme is a metalloenzyme comprising at least one divalent cation for use in treating a NMDA related condition in a subject in need thereof.

The present invention also relates to an enzyme having an anticholinergic activity, wherein said enzyme is a metalloenzyme comprising at least one divalent cation for use in treating an acetylcholine related condition in a subject in need thereof.

In one embodiment, said at least one divalent cation is selected from the list comprising Zn²⁺, Mg²⁺, Ni²⁺, Cd²⁺, Mn²⁺, Co²⁺, Fe²⁺, and Ag²⁺, preferably is Zn²⁺. In one embodiment, said enzyme is selected from the group comprising phosphotriesterases and phosphotriesterases derivatives. Preferably, said enzyme is OPD or an OPD derivative, preferably said enzyme is SEQ ID NO: 1, SEQ ID NO: 2 or SEQ ID NO: 53.

In one embodiment, said enzyme has an anticholinergic activity. In one embodiment, said enzyme has a phosphotriesterase activity. In one embodiment, said enzyme is a phosphotriesterase derivative and is not capable of hydrolyzing an organophosphorous molecule, preferably phosmet and/or fenthion.

The present invention also relates to a pharmaceutical composition comprising the enzyme for use as hereinabove described, in combination with at least one pharmaceutically acceptable excipient.

The present invention also relates to a medicament comprising the enzyme for use as hereinabove described.

In one embodiment of the invention, the NMDA related disease, disorder or condition is pain. In one embodiment, pain is hyperalgesia, such as, for example, opioid-induced hyperalgesia, hyperalgesia induced by other analgesics, preferably analgesics acting on the glutamate neurotransmission, or hyperalgesia induced by a chemotherapeutic agent or any other drug. In another embodiment, pain is neuropathy-associated pain, such as, for example, pain associated with neuropathy induced by a chemotherapeutic treatment, drug-induced neuropathy, or psychiatric medication induced neuropathy. In another embodiment, pain is associated with excitotoxicity, preferably with glutamate excitotoxicity, and/or is associated with malfunctioning of glutamatergic neurotransmission. In another embodiment, pain is associated with chronic brain impairment.

In another embodiment, the NMDA related condition is glutamate excitotoxicity.

In another embodiment, the NMDA related condition is blepharospasm, tinnitus and pathologic hiccup.

In one embodiment, said acetylcholine related condition is selected from the group comprising spinal or central spasticity, Alzheimer' s disease, schizophrenia, psychoses, Obsessive-compulsive disorder (OCD), opioid dependence, cocaine dependence, pathologic gambling, pervasive development disorders, Schwartz-Jampel Syndrome, blepharospasm, tinnitus and pathologic hiccup.

The present invention also relates to a medical device coated with an enzyme having a NMDA antagonist activity, wherein said enzyme is a metalloenzyme comprising at least one divalent cation.

Another object of the invention is a coating composition comprising with an enzyme having a NMDA antagonist activity, wherein said enzyme is a metalloenzyme comprising at least one divalent cation.

### Definitions

In the present invention, the following terms have the following meanings:
“Phosphotriesterase” refers to an enzyme capable of hydrolyzing phosphotriesters. The enzyme pocket is composed with three subsites each one binding R1, R2, R3, the alkyl residus of the phosphotriester.
“Phosphomonoesterase” refers to an enzyme capable of hydrolyzing the phosphoester bound (P—O—C) of organophosphorus compound: the phosphomonoesters (R—O—PO₃H₂).
An “anticholinergic agent” refers to a compound capable of inhibiting an acetylcholine related pathway. In one embodiment, an anticholinergic agent may inhibit the activity of a cholinergic receptor, such as, for example, a nicotinic and/or a muscarinic acetylcholine receptor. In another embodiment, an anticholinergic agent may activate the acetylcholinesterase enzyme, and thus induce the degradation of acetylcholine within the synaptic cleft. Preferably, in the present invention, an anticholinergic agent activates the acetylcholinesterase enzyme. Accordingly, the term “anticholinergic activity” refers to the activity of inhibiting an acetylcholine related pathway, preferably of activating the acetylcholinesterase enzyme.
“Treating” refers to both therapeutic treatment and prophylactic or preventative measures; wherein the object is to prevent or slow down (lessen) the target disease, disorder or condition. Those in need of treatment include those already with the disease, disorder or condition as well as those prone to have the target disease, disorder or condition or those in whom the target disease, disorder or condition is to be prevented. A subject or mammal is successfully “treated” for a disease, disorder or condition if, after receiving a therapeutic amount of an enzyme of the present invention, the subject shows observable and/or measurable reduction in or absence of one or more of the following: reduction in the number of pathogenic cells; reduction in the percent of total cells that are pathogenic; and/or relief to some extent, one or more of the symptoms associated with the specific disease, disorder or condition; reduced morbidity and mortality, and improvement in quality of life issues. The above parameters for assessing successful treatment and improvement in the disease are readily measurable by routine procedures familiar to a physician.
“Therapeutically effective amount” means level or amount of enzyme that is aimed at, without causing significant negative or adverse side effects to the target, (1) delaying or preventing the onset of the target disease, disorder, or condition; (2) slowing down or stopping the progression, aggravation, or deterioration of one or more symptoms of the target disease, disorder, or condition; (3) bringing about ameliorations of the symptoms of the target disease, disorder, or condition; (4) reducing the severity or incidence of the target disease, disorder, or condition; or (5) curing the target disease, disorder, or condition. A therapeutically effective amount may be administered prior to the onset of the target disease, disorder, or condition, for a prophylactic or preventive action. Alternatively or additionally, the therapeutically effective amount may be administered after initiation of the target disease, disorder, or condition, for a therapeutic action.
“Pharmaceutically acceptable excipient” refers to an excipient that does not produce an adverse, allergic or other untoward reaction when administered to a subject. It includes any and all solvents, dispersion media, coatings, antibacterial and antifungal agents, isotonic and absorption delaying agents and the like. For human administration, preparations should meet sterility, pyrogenicity, general safety and purity standards as required by FDA Office of Biologics standards.
“Subject” refers to an animal, preferably a mammal, more preferably a human.
“About” preceding a figure means plus or less 10% of the value of said figure.

### DETAILED DESCRIPTION

The present invention relates to an enzyme having a NMDA antagonist activity, wherein said enzyme is a metalloenzyme comprising at least one divalent cation, preferably at least one Zn²⁺ ion. In one embodiment, the enzyme of the invention comprises one divalent cation, preferably one Zn²⁺ ion. In another embodiment, the enzyme of the invention comprises two divalent cations, preferably two Zn²⁺ ions.

In one embodiment, said enzyme is selected from the group comprising phosphotriesterases and phosphotriesterases derivatives.

Methods for measuring in vitro the NMDA antagonist activity of enzymes are well known from the skilled artisan. Examples of such methods include, but are not limited to, patch clamp experiment with a solution of NMDA receptors purified and reconstituted in lipid bilayers. In one embodiment, the external medium is provided at the same composition within the intersynaptic cleft. Successively, glutamate and Zn²⁺ and Mg²⁺ will be added to record any electric activity as an evidence of NMDA residual activity. The inhibition of NMDA receptor will thus induce a lack of electric activity (0 mA +/−standard deviation error of the patch clamp materials).

In one embodiment, the NMDA antagonist activity of the enzyme of the invention is measured by electrophysiology, as shown in Example 3. In one embodiment, said measurement is performed on dorsal root ganglion nociceptor neurons of rats.

In one embodiment, the enzyme of the invention, when used at 500 nM, inhibits the NMDA current by at least 50%, preferably at least 60, 70, 80, 90, 91, 92, 93, 94, 95, 96, 97, 98, 99% or more.

In another embodiment, the enzyme of the invention, when used at 50 nM, inhibits the NMDA current by at least 10%, preferably at least 10, 15, 20, 25, 30, 35, 40, 45, 50, 55, 60, 70, 80, 90, 95% or more.

Another object of the invention is an enzyme having an anticholinergic activity, wherein said enzyme is a metalloenzyme comprising at least one divalent cation, preferably at least one Zn²⁺ ion. In one embodiment, the enzyme of the invention comprises one divalent cation, preferably one Zn²⁺ ion. In another embodiment, the enzyme of the invention comprises two divalent cations, preferably two Zn²⁺ ions.

In one embodiment, said enzyme is selected from the group comprising phosphotriesterases and phosphotriesterases derivatives. Preferably, the enzyme is an activator of the acetylcholinesterase enzyme.

Methods for measuring in vitro the anticholinergic activity of enzymes are well known from the skilled artisan. Examples of such methods include, but are not limited to measuring the hydrolysis of acetylcholine in choline and acetate which interacts with DTNB and will be evidenced by spectrophotometry UV visible. In vivo methods by coupling organotypic slices with multi-electrode array could be used in addition to evidence an acetylcholine esterase effect (parasympatholytic effect) versus reference (atropin).

Another object of the invention is an enzyme having both a NMDA antagonist activity and an anticholinergic activity, wherein said enzyme is a metalloenzyme comprising at least one divalent cation, preferably at least one Zn²⁺ ion. In one embodiment, the enzyme of the invention comprises one divalent cation, preferably one Zn²⁺ ion. In another embodiment, the enzyme of the invention comprises two divalent cations, preferably two Zn²⁺ ions. In one embodiment, said enzyme is selected from the group comprising phosphotriesterases and phosphotriesterases derivatives.

In one embodiment of the invention, the enzyme of the invention is a natural metalloenzyme, preferably a natural phosphotriesterase, i.e. a metalloenzyme or phosphotriesterase naturally expressed by a non-genetically modified living organism. Examples of living organisms that may naturally express phosphotriesterase include, but are not limited to, bacteria (such as, for example, Pseudomonas diminuta (also known as Brevundimonas diminuta), Flavobacterium sp. ATCC 27551, Escherichia coli, Mycobacterium tuberculosis, Mycoplasma pneumoniae or Agrobacterium radiobacter), archae (such as, for example, Sulfolobus solfataricus or Sulfolobus acidocaldarius), fungi, vertebrates (such as, for example, mammal, rat, mouse or human), insects (such as, for example, Musca domestica, Lucilia cuprina or Drosophila melanogaster).

Examples of natural phosphotriesterases include, but are not limited to, OPH (also referred as OPD) expressed by P. diminuta or Flavobacterium sp. ATCC 27551 (SEQ ID NO: 1), OPDA expressed by A. radiobacter (SEQ ID NO: 3), ePHP expressed by E. coli (SEQ ID NO: 5), mtPHP expressed by Mycobacterium tuberculosis (SEQ ID NO: 6), mpPHP expressed by Mycoplasma pneumonia, organophosphorous hydrolase expressed by Sphingomonas sp. JK1, or parathion hydrolase expressed by Chryseobacterium balustinum (SEQ ID NO: 7), phosphotriesterases expressed by Sulfolobus solfataricus (SEQ ID NO: 8) or Sulfolobus acidocaldarius (SEQ ID NO: 12), paraoxanases (PON1) expressed by mammals (such as, for example, human paraoxanase SEQ ID NO: 16), or phosphotriesterases expressed by insects (such as, for example, Musca domestica (SEQ ID NO: 17), Lucilia cuprina or Drosophila melanogaster (SEQ ID NO: 18)).

In one embodiment of the invention, the enzyme of the invention is derived from a natural metalloenzyme, such as, for example, a natural phosphotriesterase, i.e. a phosphotriesterase naturally expressed by a non-genetically modified living organism. Examples of living organisms that may express phosphotriesterase include, but are not limited to, bacteria (such as, for example, Pseudomonas diminuta, Flavobacterium sp. ATCC 27551, Escherichia coli, Mycobacterium tuberculosis, Mycoplasma pneumoniae or Agrobacterium radiobacter), archae (such as, for example, Sulfolobus solfataricus or Sulfolobus acidocaldarius), fungi, vertebrates (such as, for example, mammal, rat, mouse or human), insects (such as, for example, Musca domestica, Lucilia cuprina or Drosophila melanogaster).

As used herein, the term “derived” refers to an enzyme that typically differs from an enzyme from which it derives in one or more substitutions, deletions, additions and/or insertions. Such derived enzymes may be naturally occurring or may be synthetically generated, for example, by modifying one or more of the polynucleotide sequences encoding the original enzyme and evaluating one or more biological activities of the encoded polypeptide as described herein and/or using any of a number of techniques well known in the art.

In one embodiment of the invention, the amino acid sequence of the derived metalloenzyme, preferably phosphotriesterase of the invention has at least about 50%, preferably at least about 60%, more preferably at least about 70, 75, 80, 85, 90, 91, 92, 93, 94, 95, 96, 97, 98, or 99% identity with the amino acid sequence of the phosphotriesterase from which it derives.

As used herein, the term “identity”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer

Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

In one embodiment of the invention, the enzyme of the invention is, or is derived from, a hyperthermophilic phosphotriesterase. As used herein, a “hyperthermophilic phosphotriesterase” refers to a phosphotriesterase that is expressed by a hyperthermophile organism, wherein a hyperthermophile organism is a living organism capable of living (especially of developing, growing and dividing) at a temperature of more than about 60° C., preferably of more than about 80° C. Preferably, a hyperthermophilic phosphotriesterase thus has a phosphotriesterase activity at a temperature of more than about 60° C., preferably of more than about 80° C. Examples of hyperthermophilic phosphotriesterases include, but are not limited to, phosphotriesterases expressed by Sulfolobus solfataricus (SEQ ID NO: 8) or Sulfolobus acidocaldarius (SEQ ID NO: 12).

In another embodiment of the invention, the enzyme of the invention is, or is derived from, a mesophilic phosphotriesterase. As used herein, a “mesophilic phosphotriesterase” refers to a phosphotriesterase that is expressed by a mesophilic organism, wherein a mesophilic organism is a living organism capable of living (especially of developing, growing and dividing) at a moderate temperature, preferably at a temperature ranging from about 20° C. to about 45° C. Preferably, a mesophilic phosphotriesterase thus has a phosphotriesterase activity at a moderate temperature, preferably at a temperature ranging from about 20° C. to about 45° C. Examples of mesophilic phosphotriesterase include, but are not limited to, OPH expressed by P. diminuta or Flavobacterium sp. ATCC 27551 (SEQ ID NO: 1), OPDA expressed by A. radiobacter (SEQ ID NO: 3), ePHP expressed by E. coli (SEQ ID NO: 5), mtPHP expressed by Mycobacterium tuberculosis (SEQ ID NO: 6), mpPHP expressed by Mycoplasma pneumonia, organophosphorous hydrolase expressed by Sphingomonas sp. JK1, or parathion hydrolase expressed by Chryseobacterium balustinum (SEQ ID NO: 7).

In another embodiment of the invention, the enzyme is, or is derived from, a phosphotriesterase expressed by an animal, such as, for example, paraoxanases (PON1) expressed by mammals (such as, for example, human paraoxanase SEQ ID NO: 16), or phosphotriesterases expressed by insects (such as, for example, Musca domestica (SEQ ID NO: 17), Lucilia cuprina or Drosophila melanogaster (SEQ ID NO: 18)).

In another embodiment of the invention, the enzyme of the invention is, or is derived from, a mutant phosphotriesterase such as, for example, the mutant phosphotriesterases described in EP 1 392 825, WO2005/059125, US2006/154329, Yang et al (Protein

Engineering, 16(2), 135-145, 2003), Ely et al (Biochem J. 2010, 432, 565-573) and EP 2 142 644, which are incorporated in their entirety by reference.

Mutant phosphotriesterases described in EP 1 392 825 include, but are not limited to, mutants of OPDA from A. radiobacter (SEQ ID NO: 3), comprising the following mutations: P42S, P1345, A1705 and S237G; Al 19D; F272L and/or Y257H. Specific examples of mutants of OPDA from A. radiobacter described in EP 1 392 825 include, but are not limited to, SEQ ID NO: 19 and 21.

Mutant phosphotriesterases described in WO2005/059125 include, but are not limited to, mutants of OPDA from A. radiobacter (SEQ ID NO: 3), comprising the following mutations: P42S; A119D; A119H; A119Y; A119E; A119K; A1191; A119V; A119G; A119R; A119C; A119L; W130F; F131A; S237G; F305A; Y308F; Y308L; Y308S; Y308G; Y308A; A119H et Y308F; P134S and A170S; P42S and S237G; A119H and W130F; P134S and A170S and S237G; P42S and P134S and A170S; and/or P42S, P134S, A170S and S237G. Specific examples of mutants of OPDA from A. radiobacter described in WO2005/059125 include, but are not limited to, SEQ ID NO: 19, 21 and 23.

Mutant phosphotriesterases described in US2006/154329 include, but are not limited to, mutants of OPDA from A. radiobacter (SEQ ID NO: 3), wherein the sequence of the signal peptide (comprising amino acids 1 to 28 of SEQ ID NO: 3) is replaced by the following signal peptide:

M-X1-K/R-X2-K/R-X3-RR-X4-K/R-A

in which X1 is a sequence of 0 to 10 amino acids; X2 is a sequence of 0 to 3 amino acids; X3 is a sequence of 0 to 10 amino acids; and X4 is a sequence of 15 to 24 amino acids in which at least 75% up to about 90% of the residues are hydrophobic. Specific examples of signal peptides described by US2006/154329 include, but are not limited to, SEQ ID NO: 25 to 52.

Mutant phosphotriesterases described in Yang et al include, but are not limited to, mutants of OPH expressed by P. diminuta and Flavobacterium sp. ATCC 27551 (SEQ ID NO: 1) comprising the following mutations: H254R; I274T; T352A; K185R; D208G; Q211L; N265D; K285R; G348C; and/or K294N.

Mutant phosphotriesterases described in Ely et al include, but are not limited to, mutants of OPDA from A. radiobacter (SEQ ID NO: 3), comprising the following mutations: Y257F and/or R254H.

Mutant phosphotriesterases described in EP 2 142 644 include, but are not limited to, mutants of the phosphotriesterase enzyme expressed by Sulfolobus solfataricus (SEQ ID NO: 8), comprising substitutions of the following residues: Y97; Y99; R223 and/or C258 and optionally substitutions of the following residues: V27; P67; T68; L72; D141; G225; L226; F229; W263; W278; V27, L72, D141, G225 and L226; and/or P67, T68, F229, W263 and/or W278; and mutants of the phosphotriesterase enzyme expressed by Sulfolobus acidocaldarius (SEQ ID NO: 12), comprising substitutions of the following residues: Y98; Y100; R224 and/or C259 and optionally substitutions of the following residues: V28; P68; T69; L73; D142; G226; L227; F230; W264; W279; V28, L73, D142, G226 and L227; and/or P68, T69, F230, W264 and/or W279. Specific examples of mutants of the phosphotriesterase enzyme expressed by Sulfolobus solfataricus include, but are not limited to, SEQ ID NO: 9, 10 and 11. Specific examples of mutants of the phosphotriesterase enzyme expressed by Sulfolobus acidocaldarius include, but are not limited to, SEQ ID NO: 13, 14 and 15.

In one embodiment of the invention, the enzyme is, or is derived from, SEQ ID NO: 1, 3, 5-19; 21; 23 or 24. Preferably, the enzyme of the invention is, or is derived from, SEQ ID NO: 1.

In one embodiment of the invention, the enzyme is, or is derived from, SEQ ID NO: 1, 3, 5-19; 21; 23 or 24 wherein the signal peptide is deleted.

In one embodiment, the enzyme is, or is derived from, SEQ ID NO: 2, wherein SEQ ID NO: 2 corresponds to SEQ ID NO: 1 wherein the signal peptide (amino acids 1 to 29 of SEQ ID NO: 1) sequence is deleted. In one embodiment, the enzyme is SEQ ID NO: 53, corresponding to SEQ ID NO: 2 further comprising a methionine in N-term.

In one embodiment, the enzyme is, or is derived from, SEQ ID NO: 4, wherein SEQ ID NO: 4 corresponds to SEQ ID NO: 3 wherein the signal peptide (amino acids 1 to 28 of SEQ ID NO: 3) sequence is deleted.

In one embodiment, the enzyme is, or is derived from, SEQ ID NO: 20, wherein SEQ ID NO: 20 corresponds to SEQ ID NO: 19 wherein the signal peptide (amino acids 1 to 28 of SEQ ID NO: 19) sequence is deleted.

In one embodiment, the enzyme is, or is derived from, SEQ ID NO: 22, wherein SEQ ID NO: 22 corresponds to SEQ ID NO: 21 wherein the signal peptide (amino acids 1 to 28 of SEQ ID NO: 21) sequence is deleted.

In one embodiment, the enzyme is, or is derived from, SEQ ID NO: 24, wherein SEQ ID NO: 24 corresponds to SEQ ID NO: 23 wherein the signal peptide (amino acids 1 to 28 of SEQ ID NO: 23) sequence is deleted.

In one embodiment of the invention, the enzyme is, or is derived from, SEQ ID NO: 1, 3, 5-19; 21; 23 or 24 wherein the signal peptide is replaced by a heterologous signal peptide. Examples of heterologous signal peptides are described, for example, in US2006/154329 and include, without limitation, SEQ ID NO: 25 to 52.

PTE are metalloenzymes, comprising two catalytic ions. According to the invention, the enzyme is the holoenzyme, as the inventors demonstrated that the apoenzyme (i.e. the enzyme that does not comprise the catalytic ions) does not possess any NMDA antagonist activity.

In one embodiment, the holoenzyme comprises two divalent cations, wherein at least one of the divalent cations is selected from the group comprising Zn²⁺, Mg²⁺, Ni²⁺, Cd²⁺, Mn²⁺, Co²⁺, Fe²⁺, and Ag²⁺, preferably, at least one of the divalent cations is Zn²⁺.

In one embodiment, the holoenzyme comprises two divalent cations selected from the group comprising Zn²⁺, Mg²⁺, Ni²⁺, Cd²⁺, Mn²⁺, Co²⁺, Fe²⁺, and Ag²⁺. Preferably, the enzyme of the invention comprises Zn²⁺/Zn²⁺, Zn²⁺/Co²⁺, Zn²⁺/Mg²⁺, Co²⁺ /Mg²⁺ or Mg²⁺/Mg²⁺.

Methods for changing the catalytic ions of metalloenzymes are well known from the skilled artisan, and include, without limitation, dialyze.

In one embodiment of the invention, the enzyme is SEQ ID NO: 53, and the enzyme is a holoenzyme comprises two divalent cations, wherein at least one of the divalent cations is selected from the group comprising Zn^{2+, Mg}²⁺, Ni²⁺, Cd²⁺, Mn²⁺, Co²⁺, Fe²⁺, and Ag²⁺, preferably, at least one of the divalent cations is Zn²⁺.

In one embodiment of the invention, the enzyme of the invention has a phosphotriesterase activity. Methods for measuring the phosphotriesterase activity of an enzyme are well-known from the skilled artisan. Examples of such methods include, but are not limited to measuring the hydrolysis of paraoxon which produce p-nitrophenol molecule providing an absorbance maximum at 405 nm and followed by spectrophotometric method.

In one embodiment, the invention thus relates to an enzyme having a NMDA antagonist activity, wherein said enzyme is a phosphotriesterase, or a phosphotriesterase derivative having a phosphotriesterase activity. In another embodiment, the invention thus relates to an enzyme having an anticholinergic activity, wherein said enzyme is a phosphotriesterase, or a phosphotriesterase derivative having a phosphotriesterase activity. In another embodiment, the invention thus relates to an enzyme having both a NMDA antagonist activity and an anticholinergic activity, wherein said enzyme is a phosphotriesterase, or a phosphotriesterase derivative having a phosphotriesterase activity.

In another embodiment, the enzyme of the invention has a phosphomonoesterase activity. Methods for measuring the phosphomonoesterase of an enzyme are well-known from the skilled artisan. Examples of such methods include, but are not limited to measuring phosphatase standardized activities such as the dealkylation of an akyl chain on the phosphorus atom (see for example, in Masson & Rochu, Acta naturae, 2009).

In one embodiment, the invention thus relates to an enzyme having a NMDA antagonist activity, wherein said enzyme is a phosphotriesterase derivative having a phosphomonoesterase activity. In another embodiment, the invention thus relates to an enzyme having an anticholinergic activity, wherein said enzyme is a phosphotriesterase derivative having a phosphomonoesterase activity. In another embodiment, the invention thus relates to an enzyme having both a NMDA antagonist activity and an anticholinergic activity, wherein said enzyme is a phosphotriesterase derivative having a phosphomonoesterase activity.

In another embodiment, the enzyme of the invention is not capable of hydrolyzing an organophosphorous molecule. Preferably, the enzyme is not capable of hydrolyzing phosmet and/or fenthion. Methods for measuring the hydrolysis of an organophosphorous molecule, preferably phosmet and/or fenthion, by an enzyme, are well-known from the skilled artisan. Examples of such methods include, but are not limited to measuring phosphotriesterase activity as for enzymes capable of hydrolyzing phosphotriesters (see above).

In one embodiment, the invention thus relates to an enzyme having a NMDA antagonist activity, wherein said enzyme is a phosphotriesterase derivative that is not capable of hydrolyzing an organophosphorous molecule, preferably phosmet and/or fenthion. In another embodiment, the invention thus relates to an enzyme having an anticholinergic activity, wherein said enzyme is a phosphotriesterase derivative that is not capable of hydrolyzing an organophosphorous molecule, preferably phosmet and/or fenthion. In another embodiment, the invention thus relates to an enzyme having both a NMDA antagonist activity and an anticholinergic activity, wherein said enzyme is a phosphotriesterase derivative that is not capable of hydrolyzing an organophosphorous molecule, preferably phosmet and/or fenthion.

In one embodiment, the enzyme of the invention is obtained by a cloning method, such as, for example, using any production system known in the art, such as, for example, bacterial (such as, for example, E. coli), yeast, baculovirus-insect cell, or mammalian cells such as HEK or CHO, expression system.

In another embodiment wherein the enzyme is a natural metalloenzyme, preferably a natural phosphotriesterase, the enzyme is obtained from the non-genetically modified living organism producing it.

In one embodiment, the enzyme of the invention is isolated. As used herein, an “isolated enzyme” is one that has been separated and/or recovered from a component of its natural environment. Contaminant components of its natural environment are materials that would interfere with uses of the enzyme, and may include other enzymes, hormones, and other proteinaceous or nonproteinaceous components. In preferred embodiments, the enzyme is purified: (1) to greater than 95% by weight of enzymes as determined by the Lowry method, and most preferably more than 99% by weight; (2) to a degree sufficient to obtain at least 15 residues of N-terminal or internal amino acid sequence by use of a spinning cup sequenator; or (3) to homogeneity as shown by SDS-PAGE under reducing or non-reducing conditions and using Coomassie blue or, preferably, silver staining. Isolated enzymes include the enzyme in situ within recombinant cells since at least one component of the enzyme's natural environment will not be present. Ordinarily, however, isolated enzyme will be prepared by at least one purification step.

The present invention also relates to a composition comprising an enzyme of the invention.

Preferably, the enzyme, or the composition comprising the enzyme of the invention possesses a low content of endotoxins. In some embodiments, the enzyme or the composition comprising the enzyme of the invention possesses an endotoxin level of less than 1 EU/mg, less than 0.50 EU/mg, less than 0.20 EU/mg, or less than 0.15 EU/mg.

The present invention also relates to a pharmaceutical composition comprising an enzyme of the invention in association with at least one pharmaceutically acceptable excipient. In one embodiment, the pharmaceutical composition of the invention comprises the composition of the invention.

The present invention also relates to a medicament comprising an enzyme of the invention. In one embodiment, the medicament of the invention comprises the composition or the pharmaceutical composition of the invention.

In one embodiment of the invention, the composition, pharmaceutical composition or medicament of the invention comprises an amount of the enzyme of the invention ranging from about 1 nM to about 1 mM, preferably from about 10 nM to about 250 μM, more preferably from about 50 nM to about 2.5 μM.

In an embodiment, the enzyme, or the composition, pharmaceutical composition or medicament of the invention is orally administered. In this embodiment, oral preparations include tablets, capsules, powders, granules, and syrups. According to a first embodiment, the form adapted to oral administration is a solid form selected from the group comprising tablets, pills, capsules, soft gelatin capsules, sugarcoated pills, orodispersing/orodispersing tablets, effervescent tablets or other solids. According to a second embodiment, the form adapted to oral administration is a liquid form, such as, for example, a drinkable solution, liposomal forms and the like.

In another embodiment, the enzyme, or the composition, pharmaceutical composition or medicament of the invention is systemically administered.

In another embodiment, the enzyme, or the composition, pharmaceutical composition or medicament of the invention is parenterally administered, for example by intravenous injection, intramuscular injection, subcutaneous injection, intradermic injection, intraperitoneal injection, intracerebroventrocular (ICV) infusion, intracisternal injection, intrathecal injection, epidural injection or infusion. According to this embodiment, the enzyme or the composition, pharmaceutical composition or medicament of the invention is in a form adapted for injection, preferably selected from the group comprising solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

In another embodiment, the enzyme, or the composition, pharmaceutical composition or medicament of the invention is topically administered. Examples of topical administrations include, but are not limited to, sublingual administration or dermal administration. Examples of forms adapted to topical administrations include, but are not limited to, ointment, paste, cream, gel, liposomal forms, patches, such as, for example, transdermal patches, or mucoadhesive patches (such as, for example, mucoadhesive buccal patches).

In another embodiment, the enzyme, or the composition, pharmaceutical composition or medicament of the invention is administered by the respiratory tract, such as, for example, by inhalation spray, nasal spray, aerosol and the like. In one embodiment, the enzyme, or the composition, pharmaceutical composition or medicament of the invention is inhaled.

In another embodiment, the enzyme, or the composition, pharmaceutical composition or medicament of the invention is rectally administered. Examples of forms adapted to rectal administration include, but are not limited to, suppositories, rectal capsules, rectal gels, rectal foams or rectal ointments.

The present invention also relates to an enzyme of the invention, or a composition, pharmaceutical composition or medicament for, or for use in, treating a disease, disorder or condition of the central nervous system in a subject in need thereof.

The present invention also relates to a method for treating a disease, disorder or condition of the central nervous system in a subject in need thereof, wherein said method comprises administering to the subject an enzyme of the invention.

In one embodiment of the invention, a therapeutically effective amount of the enzyme of the invention is administered to the subject. In one embodiment, the enzyme is comprised in a composition, pharmaceutical composition or medicament of the invention.

In an embodiment, the therapeutically effective amount of an enzyme of the invention may be appropriately determined in consideration of, for example, the age, weight, sex, difference in diseases, and severity of the condition of individual subject. It will be understood that the specific dose level and frequency of dosage for any particular subject may be varied and will depend upon a variety of factors including the activity of the enzyme employed, the metabolic stability and length of action of that enzyme, the age, body weight, general health, sex, diet, mode and time of administration, rate of excretion, drug combination, the severity of the particular condition, and the host undergoing therapy.

In one embodiment, the subject is affected by, preferably is diagnosed with, a disease, disorder or condition of the central nervous system. In another embodiment, the subject is at risk of developing a disease, disorder or condition of the central nervous system.

Examples of risk factors include, but are not limited to, predisposition to a disease, disorder or condition of the central nervous system, such as, for example, familial or genetic predisposition; environmental conditions, medical treatment, surgical operation, exposure to an anesthetic agents or lifestyle.

Examples of diseases, disorders or conditions of the central nervous system include, but are not limited to, NMDA related diseases, disorders or conditions; acetylcholine related diseases, disorders or conditions, and/or autonomous nervous system related diseases, disorders or conditions.

The present invention also relates to a method for inhibiting a NMDA receptor, comprising administering an enzyme of the invention.

The present invention also relates to an enzyme of the invention, having a NMDA antagonist activity and optionally an anticholinergic activity, for, or for use in, neuroprotection in a subject.

The present invention also relates to a method of providing neuroprotection in a subject in need thereof, comprising administering to the subject an enzyme of the invention.

As used herein, the term “neuroprotection” refers to preventing or slowing the development of neurologic disorders such as, for example, disorders of the central nervous system. In one embodiment, neuroprotection may aim at stopping or slowing down the loss of neurons related to these diseases.

The present invention thus also relates to an enzyme of the invention or a composition, pharmaceutical composition or medicament of the invention, having a NMDA antagonist activity and optionally an anticholinergic activity, for, or for use in, treating a NMDA related disease, disorder or condition in a subject.

The present invention also relates to a method for treating a NMDA related disease, disorder or condition in a subject in need thereof, comprising administering to the subject an enzyme of the invention.

In one embodiment of the invention, a therapeutically effective amount of the enzyme of the invention is administered to the subject. In one embodiment, the enzyme is comprised in a composition, pharmaceutical composition or medicament of the invention.

As used herein, the term “NMDA related disease, disorder or condition” includes all medical conditions alleviated by treatment with an NMDA antagonist. This term includes all diseases, disorders or conditions that are acknowledged now, or that will be found in the future, to be associated with the NMDA receptor activity.

In one embodiment, the NMDA related disease, disorder or condition is pain. Therefore, according to one embodiment, the present invention relates to an enzyme of the invention or a composition, pharmaceutical composition or medicament of the invention, for, or for use in, treating pain in a subject in need thereof. Moreover, the present invention also relates to a method for treating pain in a subject in need thereof, comprising administering to the subject an enzyme of the invention.

Examples of pain include, but are not limited to, acute pain, chronic pain, allodynia, hyperalgesia, visceral pain, phantom pain, post-operative pain, neuropathic pain, peripheral neuropathy including, for example peripheral neuropathy induced by nociception, inflammation, ischemia, viral infection (HZV), traumatic and other mechanical nerve injury, cancer, diabetes mellitus, HIV infection, fibromyalgia, trigeminus neuralgia, inflammatory bowel diseases (IBD), irritative bowel syndrome (IBS), arthritis including rheumatoid arthritis, osteoarthritis (degenerative joint disease), multiple sclerosis (MS) and gout (metabolic arthritis).

In one embodiment, pain is hyperalgesia. As used herein, the term “hyperalgesia” refers to an increased sensitivity to pain. Hyperalgesia may result from damages to nociceptors or to peripheral nerves. Example of hyperalgesia include, but are not limited to, opioid-induced hyperalgesia, hyperalgesia induced by other analgesics, preferably analgesics acting on the glutamate neurotransmission, or hyperalgesia induced by a chemotherapeutic agent or any other drug.

The repeated and prolonged use of analgesics, in particular of opiate analgesics may lead to a loss of effectiveness (tolerance) followed by hypersensitivity to pain, i.e. hyperalgesia. Examples of analgesics include, but are not limited to, morphine, fentanyl, sufentanil, alfentanyl, heroin, oxycodone, hydromorphone, levorphanol, methadone, buprenorphine, butorphanol, meperidine, and the like.

Examples of chemotherapeutic agents include, but are not limited to, procarbazine, nitrofurazone, podophyllum, mustine, ethoglucid, cisplatin, suramin, paclitaxel, chlorambucil, altretamine, carboplatin, cytarabine, docetaxel, dacarbazine, etoposide, ifosfamide with mesna, fludarabine, tamoxifen, teniposide, thioguanine, and vincristine.

Additional examples of drugs that may induce hyperalgesia include, but are not limited to, anti-microbials (such as, for example, isoniazid, ethambutol, ethionamide, nitrofurantoin, metronidazole, ciprofloxacin, chloramphenicol, thiamphenicol, diamines, colistin, streptomycin, nalidixic acid, clioquinol, sulphonamides, amphotericin, and penicillin), anti-neoplastic agents (such as, for example, procarbazine, nitrofurazone, podophyllum, mustine, ethoglucid, cisplatin, suramin, paclitaxel, chlorambucil, altretamine, carboplatin, cytarabine, docetaxel, dacarbazine, etoposide, ifosfamide with mesna, fludarabine, tamoxifen, teniposide, and thioguanine, and Vinca alkaloids, such as vincristine), cardiovascular drugs (such as, for example, propranolol, perhexiline, hydralazine, amiodarone, disopyramide, and clofibrate), hypnotics and psychotropics (such as, for example, phenelzine, thalidomide, methaqualone, glutethimide, amitriptyline, and imipramine), anti-rheumatics (such as, for example, gold, indomethacin, colchicine, chloroquine, and phenyl butazone), anti-convulsants (such as, for example, phenytoin), calcium carbimide, sulfoxone, ergotamine, propylthiouracil, sulthaime, chlorpropamide, methysergide, phenytoin, disulfiram, carbutamide, tolbutamide, methimazole, dapsone, and anti-coagulants.

In one embodiment, the enzyme of the invention decreases or blocks hyperalgesia, and/or decreases or blocks exaggerated hypersensitivity to future nociceptive stimuli.

In one embodiment, pain is neuropathy-associated pain. As used herein, the term “neuropathy” refers to damage to nerves of the peripheral nervous system. The term encompasses neuropathy of various etiologies, including, but not limited to, neuropathy caused by, resulting from, or associated with genetic disorders, metabolic/endocrine complications, diabetes, inflammatory diseases, vitamin deficiencies, malignant diseases, and toxicity, such as alcohol, organic metal, heavy metal, radiation, and drug toxicity. As used herein, the term encompasses motor, sensory, mixed sensorimotor, chronic, and acute neuropathy. As used herein the term encompasses mononeuropathy, multiple mononeuropathy, and polyneuropathy.

In one embodiment, neuropathy-associated pain is induced by a chemotherapeutic treatment. Examples of chemotherapeutic agents include, but are not limited to, procarbazine, nitrofurazone, podophyllum, mustine, ethoglucid, cisplatin, suramin, paclitaxel, chlorambucil, altretamine, carboplatin, cytarabine, docetaxel, dacarbazine, etoposide, ifosfamide with mesna, fludarabine, tamoxifen, teniposide, thioguanine, and vincristine.

In one embodiment, neuropathy-associated pain is drug-induced. Examples of drugs that may induce neuropathy include, but are not limited to, anti-microbials (such as, for example, isoniazid, ethambutol, ethionamide, nitrofurantoin, metronidazole, ciprofloxacin, chloramphenicol, thiamphenicol, diamines, colistin, streptomycin, nalidixic acid, clioquinol, sulphonamides, amphotericin, and penicillin), anti-neoplastic agents (such as, for example, procarbazine, nitrofurazone, podophyllum, mustine, ethoglucid, cisplatin, suramin, paclitaxel, chlorambucil, altretamine, carboplatin, cytarabine, docetaxel, dacarbazine, etoposide, ifosfamide with mesna, fludarabine, tamoxifen, teniposide, and thioguanine, and Vinca alkaloids, such as vincristine), cardiovascular drugs (such as, for example, propranolol, perhexiline, hydralazine, amiodarone, disopyramide, and clofibrate), hypnotics and psychotropics (such as, for example, phenelzine, thalidomide, methaqualone, glutethimide, amitriptyline, and imipramine), anti-rheumatics (such as, for example, gold, indomethacin, colchicine, chloroquine, and phenyl butazone), anti-convulsants (such as, for example, phenytoin), calcium carbimide, sulfoxone, ergotamine, propylthiouracil, sulthaime, chlorpropamide, methysergide, phenytoin, disulfiram, carbutamide, tolbutamide, methimazole, dapsone, and anti-coagulants.

In one embodiment, neuropathy-associated pain is induced by psychiatric medication, preferably by long-term treatment with psychiatric medication. Examples of psychiatric medications that may induce neuropathy and neuropathy induced pain include, but are not limited to, SSRI, antipsychotic drugs (such as, for example, haloperidol and olanzapine), benzodiazepines (such as, for example, alprazolam), lithium, stimulants, and antidepressants.

In one embodiment, pain is associated with excitotoxicity, preferably with glutamate excitotoxicity, and/or is associated with malfunctioning of glutamatergic neurotransmission. As used herein, the term “excitotoxicity” (which may also be referred as “NMDA-related neurotoxicity”) relates to a pathological process wherein nerve cells are damaged and/or killed by excessive stimulation by a neurotransmitter, preferably selected from the group comprising glutamate, aspartate, N-acetylaspartyl-glutamate, cystic acid derivatives, such as, for example, L-homocysteic acid, L-cysteinsulfonic acid, L-cysteinsulfinic acid, quinolinate; and related substances, such as, for example, NMDA, kainate, and ibotenate. Excitotoxicity may also be induced by exogenous substances, such as, for example, acromelates, domoic acid, ibotenic acid, kainate, quisqualic acid, BMAA (beta-methylamino-L-alanine), BOAA (beta-oxalylamino-L-alanine) and wilardiine, NMDA, AMPA. In one embodiment, excitotoxicity may results from overactivation of glutamate receptors, preferably NMDA receptor.

Examples of conditions associated with excitotoxicity and/or malfunctioning of glutamatergic neurotransmission include, but are not limited to, acute insults (such as, for example, cerebral ischemia, cerebral infarct, brain oedema, anoxia, inner ear insult, inner ear insult in tinnitus, head or brain or spinal cord trauma, head or brain or spinal cord injuries, trauma, sound- or drug-induced inner ear insult, ischaemia resulting from cardiac arrest or stroke or bypass operations or transplants, acute pain, hypoxia, perinatal hypoxia, and ischaemia); chronic insults (such as, for example, neurodegenerative disorders, including Morbus Huntington, Alzheimer's disease Creutzfeld-Jakob's syndrome/disease, bovine spongiform encephalopathy (BSE) prion related infections, diseases involving mitochondrial dysfunction, diseases involving [beta]-amyloid and/or tauopathy, Down's syndrome, motor neuron diseases, amyotrophic lateral sclerosis (ALS), olivoponto-cerebellar atrophy, Parkinson's disease, Neuronal Ceroid Lipofuscinosis, AIDS dementia complex, AIDS-related dementia, dementia related to HIV infections, HIV-1 encephalopathy, AIDS encephalopathy, Korsakoff syndrome, vascular dementia, and corticobasal degeneration); neurological disorders (such as, for example, tinnitus, hearing loss, sound- or drug-induced tinnitus, haloperidol-induced dyskinesias, dopaminomimetic-induced dyskinesias, chorea, Huntington's chorea, athetosis, dystonia, stereotypy, ballism, tardive dyskinesias, tic disorder, spasmodic torticollis, blepharospasm, focal and generalized dystonia, nystagmus, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, L-dopa-induced dykinesias, L-dopa-induced dykinesias in Parkinson's disease therapy, dyskinesias, dyskinesia in Huntington's disease, drug induced dyskinesias, neuroleptic-induced dyskinesias, neurodegenerative cerebellar ataxias, centrally induced neuropathic pain, convulsions, epileptic convulsions, epilepsy, temporal lobe epilepsy, myoclonic epilepsy, tremor, dementia in Alzheimer's disease, dementia in Korsakoff syndrome, dementia, hereditary cerebellar ataxias, sleep disorders, movement disorders, essential tremor, muscle spasms, and spasticity); psychological/psychiatric disorders (such as, for example, generalized anxiety disorder, obsessive-compulsive disorder, panic disorder, posttraumatic stress disorder, social phobia, phobic disorders, substance-induced anxiety disorder, delusional disorder, schizoaffective disorder, schizophreniform disorder, substance-induced psychotic disorder, delirium, post-operative cognitive deficit (POCD), cognitive impairment, learning impairment, anxiety disorders, panic disorders, anxiety and panic disorders, social anxiety disorder (SAD), attention deficit hyperactivity disorder (ADHD), attention deficit syndrome (ADS), dementia, posttraumatic stress disorder (PTSD), schizophrenia, positive or cognitive or negative symptoms of schizophrenia, major depressive disorder, major depression, depression, bipolar manic-depressive disorder, sleep disorders, agoraphobia, bulimia nervosa, eating disorders, obesity, obesity-related disorders, obesity abuse, food addiction, binge eating disorders, and hyperactivity in children; drug/alcohol abuse (such as, for example, craving (e.g., for drugs of abuse), abuse, addiction, nicotine addiction, nicotine abuse, alcohol addiction, alcohol abuse, opiate addiction, opiate abuse, cocaine addiction, cocaine abuse, amphetamine addiction, and amphetamine abuse); skin diseases (such as, for example, atopic dermatitis, itching, skin lesions induced by severe itching or atopic dermatitis, systemic sclerosis, pruritic conditions, and pruritis); diseases of the gastro-intestinal tract and metabolic diseases (such as, for example, diarrhoea, hepatic encephalopathy, hypoglycaemia, gastroesophageal reflux disease (GERD), gastrointestinal dysfunction, lower esophageal sphincter (LES) disease, functional gastrointestinal disorders, dyspepsia, vomiting, urinary incontinence, and regurgitation); diseases of the immune system (such as, for example, Sjogren's syndrome, systemic lupus erythematosus, and multiple sclerosis (MS)); eye diseases (such as, for example, eye injuries, eye diseases, eye disorders, glaucoma, retinopathy, and macular degeneration); diseases of the respiratory tract (such as, for example, respiratory tract infection, chronic laryngitis, asthma, reflux-related asthma, and lung disease); migraine; autism; restless leg syndrome (RLS); Tourette syndrome; micturition disorders; neuromuscular disorder in the lower urinary tract; and drug tolerance to opioids or to any other drug, such as, for example, nicotine, alcohol, cocaine, or amphetamine.

In one embodiment, pain is associated with chronic brain impairment. As used herein, “chronic brain impairment” relates to generalized brain dysfunction, and may be associated with the following symptoms: (i) cognitive dysfunctions, (ii) apathy or loss of energy and vitality, (iii) emotional worsening, and (iv) anosognosia (Breggin, International Journal of Risk & Safety in Medicine 23 (2011) 193-200).

In one embodiment, the NMDA related disease, disorder or condition is neuropathy, such as, for example, neuropathy caused by, resulting from, or associated with genetic disorders, metabolic/endocrine complications, diabetes, inflammatory diseases, vitamin deficiencies, malignant diseases, and toxicity, such as alcohol, organic metal, heavy metal, radiation, and drug toxicity. In one embodiment, neuropathy is induced by a chemotherapeutic treatment. In another embodiment, neuropathy is drug-induced. In another embodiment, neuropathy is induced by psychiatric medication.

In one embodiment, the NMDA related disease, disorder or condition is excitotoxicity, preferably glutamate excitotoxicity, and/or is malfunctioning of glutamatergic neurotransmission.

In one embodiment, the NMDA related disease, disorder or condition is chronic brain impairment.

Examples of NMDA related diseases, disorders or conditions include, but are not limited to, excito-toxicity, such as, for example, traumatic excito-toxicity, vascular excito-toxicity, deafness related excito-toxicity or degenerative excito-toxicity (the enzyme of the invention may thus be used for decreasing excitotoxicity related, for example, to head trauma, to cerebral ischemia and/or to deafness), Alzheimer's disease, stroke and vascular conditions such as, for example, systemic vascularitis, Crohn disease, ulcerative colitis, collagenosis disease, Polyangeitis, necrotizing glomerulonephritis, Wegener granulomatosis, Polyarteritis nodosa, Giant cell arteritis (Horton disease), Kawasaki, Henoch-Schoenlein purpura, Cryoglobulinemia, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome, Childhood disintegrative disorder, pain, such as, for example, acute pain, chronic pain, allodynia, hyperalgesia such as, for example, hyperalgesia induced by morphine treatment (such as, for example, during surgery, cancer treatment or in patients in final phase), hyperalgesia induced by opiod treatment (such as, for example, during orthopedic or digestive surgery, or in carcinology), visceral pain, phantom pain, post-operative pain, neuropathic pain, intractable neuropathic pain and other neuropathic pains (such as, for example, postherpetic neuralgia, nerve injury, the “dynias”, such as, for example, vulvodynia, phantom limb pain, root avulsions, painful diabetic neuropathy, painful traumatic mononeuropathy, painful polyneuropathy), allodynia, pain wind up, central pain syndromes (potentially caused by virtually any lesion at any level of the nervous system), and postsurgical pain syndromes (such as, for example, postmastectomy syndrome, postthoracotomy syndrome, stump pain), bone and joint pain (such as, for example, osteoarthritis), repetitive motion pain, dental pain, cancer pain, myofascial pain (such as, for example, muscular injury, fibromyalgia), perioperative pain (such as, for example, following general surgery, gynecological), chronic pain, dysmennorhea, as well as pain associated with angina, and inflammatory pain of varied origins (such as, for example, osteoarthritis, rheumatoid arthritis, rheumatic disease, teno-synovitis and gout), headache, peripheral neuropathy such as, for example peripheral neuropathy induced by nociception, inflammation, ischemia, viral infection (HZV), traumatic and other mechanical nerve injury, cancer, diabetes mellitus, HW infection, fibromyalgia, trigeminus neuralgia, inflammatory bowel diseases (IBD), irritative bowel syndrome (IBS), arthritis, such as, for example, rheumatoid arthritis, osteoarthritis (degenerative joint disease), multiple sclerosis (MS), gout (metabolic arthritis); acute insults (such as, for example cerebral ischemia, cerebral infarct, brain oedema, anoxia, inner ear insult, inner ear insult in tinnitus, head or brain or spinal cord trauma, head or brain or spinal cord injuries, trauma, sound- or drug-induced inner ear insult, ischaemia resulting from cardiac arrest or stroke or bypass operations or transplants, acute pain, hypoxia, perinatal hypoxia, and ischaemia), neurodegenerative disorders (such as, for example, Morbus Huntington, Alzheimer's disease, Creutzfeld-Jakob's syndrome/disease, bovine spongiform encephalopathy (BSE), prion related infections, diseases involving mitochondrial dysfunction, diseases involving [beta]-amyloid and/or tauopathy, Down's syndrome, motor neuron diseases, amyotrophic lateral sclerosis (ALS), olivoponto-cerebellar atrophy, Parkinson's disease, Neuronal Ceroid Lipofuscinosis, AIDS dementia complex, AIDS-related dementia, dementia related to HIV infections, HIV-1 encephalopathy, AIDS encephalopathy, Korsakoff syndrome, vascular dementia, and corticobasal degeneration), neurological disorders (such as, for example, tinnitus, hearing loss, sound- or drug-induced tinnitus, haloperidol-induced dyskinesias, dopaminomimetic-induced dyskinesias, chorea, Huntington's chorea, athetosis, dystonia, stereotypy, ballism, tardive dyskinesias, tic disorder, spasmodic torticollis, blepharospasm, focal and generalized dystonia, nystagmus, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, L-dopa-induced dykinesias, L-dopa-induced dykinesias in Parkinson's disease therapy, dyskinesias, dyskinesia in Huntington's disease, drug induced dykinesias, neuroleptic-induced dyskinesias, neurodegenerative cerebellar ataxias, centrally induced neuropathic pain, convulsions, epileptic convulsions, epilepsy, temporal lobe epilepsy, myoclonic epilepsy, tremor, dementia in Alzheimer's disease, dementia in Korsakoff syndrome, dementia, hereditary cerebellar ataxias, sleep disorders, movement disorders, essential tremor, muscle spasms, and spasticity), psychological/psychiatric disorders (such as, for example, generalized anxiety disorder, obsessive-compulsive disorder, panic disorder, posttraumatic stress disorder, social phobia, phobic disorders, substance-induced anxiety disorder, delusional disorder, schizoaffective disorder, schizophreniform disorder, substance-induced psychotic disorder, delirium, post-operative cognitive deficit (POCD), cognitive impairment, learning impairment, anxiety disorders, panic disorders, anxiety and panic disorders, social anxiety disorder (SAD), attention deficit hyperactivity disorder (ADHD), attention deficit syndrome (ADS), dementia, posttraumatic stress disorder (PTSD), schizophrenia, positive or cognitive or negative symptoms of schizophrenia, major depressive disorder, major depression, depression, bipolar manic-depressive disorder, sleep disorders, agoraphobia, bulimia nervosa, eating disorders, obesity, obesity-related disorders, obesity abuse, food addiction, binge eating disorders, and hyperactivity in children), drug/alcohol abuse (such as, for example, craving (e.g., for drugs of abuse), abuse, addiction, nicotine addiction, nicotine abuse, alcohol addiction, alcohol abuse, opiate addiction, opiate abuse, cocaine addiction, cocaine abuse, amphetamine addiction, and amphetamine abuse), skin diseases (such as, for example, atopic dermatitis, itching, skin lesions induced by severe itching or atopic dermatitis, systemic sclerosis, pruritic conditions, and pruritis), diseases of the gastro-intestinal tract and metabolic diseases (such as, for example, diarrhoea, hepatic encephalopathy, hypoglycaemia, gastroesophageal reflux disease (GERD), gastrointestinal dysfunction, lower esophageal sphincter (LES) disease, functional gastrointestinal disorders, dyspepsia, vomiting, urinary incontinence, and regurgitation), diseases of the immune system (such as, for example, Sjogren's syndrome, systemic lupus erythematosus, and multiple sclerosis (MS), eye diseases (such as, for example, eye injuries, eye diseases, eye disorders, dry eye, glaucoma, retinopathy, and macular degeneration), diseases of the respiratory tract (such as, for example, respiratory tract infection, chronic laryngitis, asthma, reflux-related asthma, and lung disease), migraine, autism, restless leg syndrome (RLS), Tourette syndrome, micturition disorders, neuromuscular disorder in the lower urinary tract, drug tolerance to opioids, depression, stroke, traumatic brain injury, neurological damage caused by epileptic seizures or by neurotoxin poisoning or by impairment of glucose and/or oxygen to the brain, vision loss caused by neurodegeneration of the visual pathway, multi-system atrophy, primary hyperalgesia, secondary hyperalgesia, primary allodynia, secondary allodynia, and other pain caused by central sensitization.

In one embodiment, the NMDA related disease, disorder or condition is selected from the group comprising hyperalgesia, such as, for example, hyperalgesia induced by morphine treatment (such as, for example, during surgery, cancer treatment or in patients in final phase), hyperalgesia induced by opiod treatment (such as, for example, during orthopedic or digestive surgery, or in carcinology), neuropathies, such as, for example, neuropathic pain, intractable neuropathic pain, allodynia, pain wind up, excitotoxicity, such as, for example, traumatic excito-toxicity, vascular excito-toxicity, deafness related excito-toxicity or degenerative excito-toxicity (the enzyme of the invention may thus be used for decreasing excitotoxicity related, for example, to head trauma, to cerebral ischemia and/or to deafness), stroke and vascular conditions such as, for example, systemic vascularitis, Crohn disease, ulcerative colitis, collagenosis disease, Polyangeitis, necrotizing glomerulonephritis, Wegener granulomatosis, Polyarteritis nodosa, Giant cell arteritis (Horton disease), Kawasaki, Henoch-Schoenlein purpura, Cryoglobulinemia, Alzheimer's disease, schizophrenia, psychoses, Obsessive-compulsive disorder (OCD), delirium, opioid dependence, cocaine dependence, pathologic gambling, pervasive development disorders, such as, for example, autism, infantile autism, Rett syndrome, Asperger syndrome and Childhood disintegrative disorder.

Preferably, the NMDA related disease, disorder or condition is selected from the group comprising hyperalgesia, such as, for example, hyperalgesia induced by morphine treatment (such as, for example, during surgery, cancer treatment or in patients in final phase), hyperalgesia induced by opiod treatment (such as, for example, during orthopedic or digestive surgery, or in carcinology), neuropathies, such as, for example, neuropathic pain, intractable neuropathic pain, allodynia, pain wind up, excitotoxicity, such as, for example, traumatic excito-toxicity, vascular excito-toxicity, deafness related excito-toxicity or degenerative excito-toxicity (the enzyme of the invention may thus be used for decreasing excitotoxicity related, for example, to head trauma, to cerebral ischemia and/or to deafness).

In one embodiment, the NMDA related disease, disorder or condition is pain associated with neuropathies, such as, for example, allodynia, excitotoxicity, such as, for example, traumatic excito-toxicity, vascular excito-toxicity, deafness related excito-toxicity or degenerative excito-toxicity (the enzyme of the invention may thus be used for decreasing excitotoxicity related, for example, to head trauma, to cerebral ischemia and/or to deafness).

More preferably, the NMDA related disease, disorder or condition is selected from the group comprising hyperalgesia, such as, for example, hyperalgesia induced by morphine treatment (such as, for example, during surgery, cancer treatment or in patients in final phase), hyperalgesia induced by opiod treatment (such as, for example, during orthopedic or digestive surgery, or in carcinology).

The present invention also relates to a method for inhibiting a cholinergic pathway, such as, for example, for inhibiting a cholinergic receptor or for activating the acetylcholinesterase enzyme, preferably for activating the acetylcholinesterase enzyme, comprising administering an enzyme of the invention.

The present invention also relates to a method for preventing post synaptic changes such as, for example, a post-synaptic degeneration, such as, for example, post synaptic changes or degeneration related to a quantitative or qualitative abnormality of acetylcholine or acetylcholinesterase into the cleft, to an increase of acetylcholine secretion into the cleft, or to dysregulation of acetylcholine secretion into the cleft.

Examples of diseases, disorders or conditions wherein such post synaptic changes or degenerations may occur include, but are not limited to, myasthenia and Schwarz-Jampel syndrome.

The present invention also relates to an enzyme of the invention, having an anticholinergic activity and optionally an NMDA antagonist activity, for, or for use in, treating an acetylcholine related disease, disorder or condition in a subject.

The present invention also relates to a method for treating an acetylcholine related disease, disorder or condition in a subject in need thereof, comprising administering to the subject an enzyme of the invention.

In one embodiment of the invention, a therapeutically effective amount of the enzyme of the invention is administered to the subject. In one embodiment, the enzyme is comprised in a composition, pharmaceutical composition or medicament of the invention.

As used herein, the term “an acetylcholine related disease, disorder or condition” includes all medical conditions alleviated by treatment with an anticholinergic agent. This term includes all diseases, disorders or conditions that are acknowledged now, or that will be found in the future, to be associated with a cholinergic pathway.

Examples of acetylcholine related diseases, disorders or conditions include, but are not limited to, spinal or central spasticity, Alzheimer's disease, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome and Childhood disintegrative disorder. Spasticity or causes of spasticity include, but are not limited to, spinal injury, post-traumatic spinal and cerebral sequels, multiple sclerosis or other demyelinating diseases (such as, for example, neuromyelitis and encephalomyelitis), myopathic syndrome, syringomyelia, encephalopathy (such as, for example, related to HIV), bladder instability and urination or micturition disorders with bladder spasticity.

In one embodiment of the invention, the acetylcholine related disease, disorder or condition is spasticity, preferably spinal or central spasticity.

In one embodiment, the acetylcholine related disease, disorder or condition is the Schwartz-Jampel Syndrome or any other orphan disease, disorder or condition related to acetylcholine.

The present invention also relates to a method for inhibiting the NMDA receptor and for inhibiting a cholinergic pathway, such as, for example, for inhibiting a cholinergic receptor or for activating the acetylcholinesterase enzyme, preferably for activating the acetylcholinesterase enzyme, comprising administering an enzyme of the invention.

The present invention also relates to an enzyme of the invention, having both an anticholinergic activity and a NMDA antagonist activity, for, or for use in, treating a NMDA and acetylcholine related disease, disorder or condition in a subject.

The present invention also relates to a method for treating a NDMA and acetylcholine related disease, disorder or condition in a subject in need thereof, comprising administering to the subject an enzyme of the invention.

In one embodiment of the invention, a therapeutically effective amount of the enzyme of the invention is administered to the subject. In one embodiment, the enzyme is comprised in a composition, pharmaceutical composition or medicament of the invention.

Examples of NMDA and acetylcholine related diseases, disorders or conditions include, but are not limited to, Alzheimer's disease, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome and Childhood disintegrative disorder.

In one embodiment, the NMDA and acetylcholine related disease, disorder or condition is pain, such as, for example, pain induced by Alzheimer's disease, schizophrenia, psychoses, Obsessive-compulsive disorder (OCD), opioid dependence, cocaine dependence, pathologic gambling, pervasive development disorders, such as, for example, autism, infantile autism, Rett syndrome, Asperger syndrome and Childhood disintegrative disorder.

The present invention also relates to an enzyme of the invention for, or for use in, treating an autonomous nervous system related disease, disorder or condition in a subject.

The present invention also relates to a method for treating an autonomous nervous system related disease, disorder or condition in a subject in need thereof, comprising administering to the subject an enzyme of the invention.

In one embodiment of the invention, a therapeutically effective amount of the enzyme of the invention is administered to the subject. In one embodiment, the enzyme is comprised in a composition, pharmaceutical composition or medicament of the invention.

As used herein, the term “an autonomous nervous system related disease, disorder or condition” includes all medical conditions that are acknowledged now, or that will be found in the future, to be associated with a dysfunction of the autonomous nervous system.

Examples of autonomous nervous system related diseases, disorders or conditions include, but are not limited to, blepharospasm, tinnitus and pathologic hiccup.

In one embodiment, the NMDA related disease, disorder or condition is an autonomous nervous system related disease, disorder or condition, such as, for example, blepharospasm, tinnitus and pathologic hiccup.

In one embodiment, the acetylcholine related disease, disorder or condition is an autonomous nervous system related disease, disorder or condition, such as, for example, blepharospasm, tinnitus and pathologic hiccup.

In one embodiment of the invention, the enzyme of the invention is administered in combination with another therapeutic compound suitable for the treatment of the target disease, disorder or condition. For example, when the method of the invention is for treating a NMDA related disease, disorder or condition, the enzyme of the invention may be administered with another NMDA antagonist. Examples of other therapeutic compounds that may be used in combination with the enzyme of the invention include, but are not limited to, opiate agonists or antagonists; NMDA antagonists; anticholinergic agents, such as, for example, memantine; alpha-2 adrenergic agonists, such as, for example, tizanidine; non-steroidal anti-inflammatory agents; COX-2 inhibitors; bradykinin Bl receptor antagonists; sodium channel blockers and antagonists; nitric oxide synthase (NOS) inhibitors; nitrous oxide; glycine site antagonists; potassium channel openers; AMPA/kainate receptor antagonists; agents decreasing the release of excitatory amino acids acting on the NMDA receptor (presynaptic action); calcium channel antagonists; GABA-A receptor modulators (such as, for example, a GABA-A receptor agonist); matrix metalloprotease (MMP) inhibitors; thrombolytic agents; opioids such as, for example, morphine; neutrophil inhibitory factor (NIF); L-Dopa; carbidopa; levodopa/carbidopa; dopamine agonists such as, for example, bromocriptine, pergolide, pramipexole, ropinirole; amantadine; catechol O-methyltransferase (COMT) inhibitors such as entacapone and tolcapone; Monoamine oxidase B (MAO-B) inhibitors; 5HT receptor agonists or antagonists; NK1 antagonists; selective serotonin reuptake inhibitors (SSRI) and/or selective serotonin and norepinephrine reuptake inhibitors (SSNRI); tricyclic antidepressant drugs, norepinephrine modulators; lithium; valproate; and neurontin (gabapentin).

The present invention also relates to the use of an enzyme of the invention for diagnosing a disease, disorder or condition of the central nervous system. The present invention also relates to a diagnostic method comprising the use of an enzyme of the invention, for diagnosing a disease, disorder or condition of the central nervous system.

The present invention also relates to a kit for measuring the inhibition of the NMDA receptor and/or for measuring the anti-cholinergic effect of a compound, wherein said kit comprises an enzyme of the invention.

The present invention also relates to a coating composition comprising an enzyme of the invention.

The present invention thus also relates to the use of an enzyme of the invention, or of a composition of the invention for coating a surface, preferably in the health sector.

Examples of surfaces that may be coated with the coating composition of the invention include, but are not limited to, tubes and catheters, prosthesis, syringes, medical instruments, biomedical devices, acupuncture needle, or surgical tools.

The present invention also relates to a medical device, a tube, a catheter, a prosthesis, a syringe, a medical instrument, an acupuncture needle, or a surgical tool coated with a composition comprising an enzyme according to the invention, or with a composition, pharmaceutical composition or medicament of the invention.

### BRIEF DESCRIPTION OF THE DRAWINGS

FIG. 1 is a combination of electrophysiological traces.

(A) Control traces, obtained during perfusion of NMDA (250 μM) for a period of 20 s, at a holding potential of −30 mV,

(B) Trace obtained during perfusion of NMDA (250 μM) for a period of 80 s. An injection of ZnCl2 (50nM, 200 μl) is applied during steady state stabilization of the NMDA current. The ZnCl2 applied alone inhibits all of the NMDA current,

(C) and (D) Traces obtained during perfusion of NMDA (250 μM). OPN-001-B (50 nM and 500 nM, respectively C and D, 200 μl) is applied during the steady state stabilization. In these two examples, OPN-001-B inhibits 33% and 100% respectively of the NMDA current, (E) Percentage inhibition of NMDA current in response to application of OPN-001-B (50 nM: 35±11% (SD) and 500nM: 92±11% (SD), n=6 respectively), OPN-001-A (500 nM, 0%, n=4) and ZnCl2 (5 nM: 0%; 50 nM: 100%, 500 nM: 100%, n =4 respectively).

### EXAMPLES

The present invention is further illustrated by the following examples.

### Example 1

### Production of the Enzyme

The amino acid sequence SEQ ID NO: 53 was cloned in a pETDuet-1 plasmid (Merck Millipore), under the control of a IPTG inducing T7 promoter, and the plasmid was used for transforming the bacterial strain E. coli BL21 DU3, to produce the OPN001-B protein, wherein OPN001-B is a holoenzyme comprising one Zn²⁺ ion and one Co²⁺ ion.

Ampicilin (present in the plasmid construct) was used to select bacteria expressing OPN001. OPN001 is induced by IPTG and purified by chromatography with ion exchange, gel filtration and desalting chromatography. At the end the yield of the purification was around 3 mg/mL.

### Preparation of the Apoenzyme OPN001-A

The protein fraction was concentrated by ultrafiltration ultrafiltration (vivaspin 6, 10 000 Da) until a final volume of 1 mL, and then dialyzed (cut-off de 3350 Da) in two successive baths of 1000 mL for 24 hours at 4° C., under gentle stirring, against a chelating solution (50 mM Hepes (pH 8.0); 150 mM NaCl; 2.5 mM EDTA; 2.5 mM beta-mercaptoethanol). The enzyme was then desalted by gel filtration on a Sephadex column (Disposable PD-10 Desalting column, GE Healthcare). The column was equilibrated with 4 CV (Column Volumes) of elution buffer (50 mM Hepes (pH 8.0), 150 mM NaCl). The protein solution supplemented with 2.5 mL of elution buffer, was then loaded onto the column. The protein fraction was then eluted with 4 ml of elution buffer into 8 fractions. The elution profile was monitored by UV spectroscopy at 280 nm. The protein fractions were pooled and concentrated at 0.150 or 0.250 mM by ultrafiltration (vivaspin 6, 10 000 Da). The protein concentration was measured by UV spectroscopy at 280 nm (Nanodrop 2000 c, thermo scientific) (ε=29575M⁻¹·cm⁻¹, 1=0.1 cm). Finally the protein solution was aliquoted and frozen in tubes in liquid nitrogen with 5% glycerol as cryoprotectant.

All stages of the preparation of OPN001-A were followed by electrophoresis: 2 μL of each fraction were deposited on SDS-PAGE gel.

### Preparation of the Holoenzyme OPN001-B

OPN001-B is the holoenzyme containing one Zn²⁺ and one Co²⁺ metallic ion. In the last purification step, the enzyme initially synthesized with Cobalt was incubated with Zn acetate at 10% of the protein concentration. The initial protein solution was concentrated to 0.250 mM or 0.150 mM by ultrafiltration (Vivaspin 6, 10 000 Da). The protein concentration was determined by UV spectroscopy at 280 nm (Nanodrop 2000 c, Thermo Scientific) (ε=29575M⁻¹·cm⁻¹, 1 =0.1cm). Then, the protein solution was aliquoted and frozen in tubes in liquid nitrogen with 5% glycerol as cryoprotectant.

All stages of the preparation of OPN001-B were followed by electrophoresis: 2 μL of each fraction were deposited on SDS-PAGE gel.

### Example 2

### Neuroprotective Effects of the Enzyme of the Invention

In this example, the effects of the enzyme of the invention (OPN-B, holoenzyme) were evaluated on glutamate intoxicated-cortical neurons in order to measure a potential neuroprotective effect, and compared to the effects of the apoenzyme (OPN-A). In parallel, the effect of these 2 enzymes was also evaluated under basal conditions (without glutamate) in order to assess a potential neurotoxic effect of the test enzymes on cortical neurons.

Glutamate is one of the principal excitatory amino acid in the central nervous system, and as such plays a crucial role in neuronal physiology. In many acute neurological conditions, there are perturbations in one or more of the normal regulatory mechanism that may lead to excessive and neurotoxic activation of glutamate receptors. Based on a primary culture of cortical neurons (isolated from rat brain), these acute conditions can be reproduced by glutamate intoxication. The impact on neurons is pointed out by morphological change occurring on neurofilament network. The potential neuroprotective/neurotoxic effects were studied in analyzing neurite network density with a specific antibody labeling (anti-heavy chain neurofilament) for mature neuron neuritis.

### Materials and Methods

### Biological Model

Cell type: Cortical neurons isolated from 15-day old Wistar rat embryos (EIS)

Culture conditions: 37° C., 5% CO₂

Culture medium: Neurobasal medium supplemented with B27 complement 2%, antibiotics (penicillin 50 U/mL—Streptomycin 50 μg/mL) and L-glutamine 2 mM.

### Test Compounds

OPN-A and OPN-B were stored at −20° C. in a liquid stock solution at 250 μM in 50 mM HEPES pH8.0, 150 mM NaCl, 5% Glycerol. For OPN-B, the stock solution is a balanced ZnAcetate solution (Zn/Protein ratio 1/10). OPN-A and OPN-B were tested at a final concentration of 2.5 μM.

### Cultures and Treatments

Cortical neurons were isolated and seeded in 96-well plates in culture medium. Cells were then incubated for 8 days in culture medium and half of the medium was changed every 2-3 days.

### Basal Conditions

At DIV8 (days in vitro 8), the culture medium was replaced by culture medium containing or not (control) the test enzymes or the solvent control (50 mM HEPES pH8.0, 150 mM NaCl) and cortical neurons were incubated for 30 hours. As a positive control condition of intoxication, glutamate was also tested in parallel (100 μM) with an incubation time of 6 hours at DIV9. All experimental conditions were performed in n=3.

Stimulated Conditions At DIVE, the culture medium was replaced by culture medium containing or not (controls) the test enzymes or the solvent control and cortical neurons were pre-incubated for 24 hours. At DIV9, the reference MK-801 (10 μM) was pre-incubated for 30 minutes before glutamate intoxication. Cortical neurons were then incubated for 6 hours with 100 μM of glutamate in presence or not (intoxicated control) of test compounds, the solvent control or the reference. A control without glutamate (non-intoxicated control) was performed in parallel. All experimental conditions were performed in n=3.

### Analysis of the Neurite Network Density

At the end of incubation, culture medium was discarded and the cells were rinsed, fixed and permeabilized. The cells were then labeled using a monoclonal anti-NF-H antibody (anti-neurofilament heavy chain, clone RT97). The primary antibody was then revealed with a fluorescent secondary antibody conjugated with Alexa Fluor® 488 (GAM-Alexa 488) and the cell nuclei were colored with Hoechst solution (bis-benzimide) in parallel.

For each condition, 10 pictures per well (3×10=30 pictures per condition) were taken using an automated cell imager, INCell Analyzer™ 1000 (GE Healthcare) with x20 lens. All the images were taken under the same conditions.

The labeling was quantified by measurement of the total surface of NF-H-positive neuritis (Integration of numerical data with the Developer Toolbox 1.5, GE Healthcare software).

### Data Management

Raw data were analyzed with Microsoft Excel® software. The inter-group comparisons were performed by Student's t-test.

Percentage of intoxication was measured as follows:

$$Intoxication ( \% ) = 100 - ( \frac{Value}{Mean of control} \times 100 )$$

Percentage of protection was measured as follows:

$$Protection ( \% ) = \frac{Intoxicated {control}^{′} s mean - Value}{Intoxicated {control}^{′} s mean - Non \text{-} intoxicated {control}^{′} s mean } \times 100$$

### Results

### Effect of the Test Enzymes Under Basal Conditions

The impact of test enzymes on neural network integrity is shown in Table 1 below.

TABLE 1

| | | Basic data | | | | |
|---|---|---|---|---|---|---|
| | | Mean NF- | | | | |
| | | H-positive | | | | |
| Treatment | | neurite | | Normalized data | | |
| Test | Concen- | surface | | Intoxication | Sem | |
| compound | tration | (μm²) | sem | (%) | (%) | p value |
| Control | — | 32605 | 1239 | 0 | 4 | — |
| Glutamate | 100 μM | 21503 | 528 | 34 | 2 | 0.0012 (*) |
| Solvent | 10% | 33150 | 1301 | −2 | 4 | 0.777 (ns) |
| control | | | | | | |
| OPN-A | 2.5 μM | 30098 | 1680 | 8 | 5 | 0.296 (ns) |
| OPN-B | 2.5 μM | 33592 | 1238 | −3 | 4 | 0.603 (ns) |

(ns): p value >0.5, Not significant
(*): p value <0.5, Significant

Treatment of cortical neurons with glutamate at 100 μM for 6 hours impacted the neuronal network integrity as observed by the significant decrease of NF-H positive neuritis (34% of intoxication). These results were expected and validated the basal part of the experiment.

At a concentration of 2.5 μM, OPN-A showed no significant effect on NF-H positive neuronal network. Moreover, the holoenzyme OPN-B, tested at 2.5 μM, and the solvent control showed no significant effect on NF-H positive neuronal network and consequently no detrimental effect on neuronal network integrity of cortical neurons.

### Effect of the Test Compounds Under Glutamate-Intoxicated Conditions

The impact of test enzymes on neural network integrity under glutamate-intoxicated conditions is shown in Table 2 below.

TABLE 2

| | | Basic data | | | | |
|---|---|---|---|---|---|---|
| | | Mean NF-H- | | | | |
| Treatment | | positive | | Normalized data | | |
| Test | | neurite | | Protection | | |
| compound | Concentration | surface (μm²) | sem | (%) | Sem (%) | p value |

Non-intoxicated conditions

| | | | | | | |
|---|---|---|---|---|---|---|
| Control | — | 32714 | 1725 | 100 | 15 | 0.0046 (*) |

Intoxicated conditions (glutamate 100 μM)

| | | | | | | |
|---|---|---|---|---|---|---|
| Control | — | 21456 | 952 | 0 | 8 | — |
| MK801 | 10 μM | 32319 | 515 | 96 | 5 | 0.0006 (*) |
| Solvent | 10% | 22130 | 1807 | 6 | 16 | 0.758 (ns) |
| control | | | | | | |
| OPN-A | 2.5 μM | 22274 | 830 | 7 | 7 | 0.553 (ns) |
| OPN-B | 2.5 μM | 18584 | 597 | 30 | 5 | 0.041 (*) |

(ns): p value >0.5, Not significant
(*): p value <0.5, Significant

Glutamate intoxication (100 μM for 6 hours) greatly impacted the neuronal network integrity of cortical neurons as shown by the significant decrease of NF-H positive neuritis. This effect was totally inhibited in presence of the NMDA receptor antagonist MK801, tested at 10 μM (˜96% of protection, p=0.0006). These results were expected and validated the stimulated part of the experiment.

The apoenzyme OPN-A, tested at 2.5 μM, and the solvent control showed no protective effect against glutamate-intoxication.

On the contrary, the holoenzyme OPN-B, tested at 2.5 μM, showed a clear protective effect against glutamate-induced NF-H positive neuritis decrease (30% of protection, p=0.041).

### Example 3

### Evaluation of the NMDA Antagonist Activity of the Enzymes of the Invention by Electrophysiology

The culture conditions of dorsal root ganglia neurons of rats were adapted from Hao and Delmas (J Neurosci. 2010, 6; 30(40)).

NMDA current recordings are performed in neurons of small and medium diameter (nociceptors). Neurons are identified visually and electrophysiologically by measuring their membrane capacitance. The nociceptors exhibit a capacitance ranging from 18 to 45 pF. These neurons express the NMDA receptor in the dorsal root ganglia. Nociceptors are specifically identified by the presence of TTX-resistant Nav1.8 sodium current. Each neuron is tested for the presence upstream of Nav1.8.

NMDA currents are low amplitude currents. Thus, electrical leakage is carefully checked during recording. Infusion or injection of products was performed close to neurons to avoid changes in concentration (dilution effect). Media infusion was extemporaneously daily handled. Products OPN-001 were diluted from stock solutions and stored at 4° C. The data were collected using an Axopatch amplifier 200-B, the Clampex software (PClamp V10, Molecular Device) and stored and analyzed using Clampfit.

### Test Compounds

| Test | Aspect/ | | |
|---|---|---|---|
| compound | Storage | Stock solution | Test concentration |
| OPN-001-A | Liquid | 150 μMol in 50 mM HEPES | 0.51 μM |
| (apoenzyme) | Storage at | pH 8.0; 150 mM NaCl 5%; | |
| | −20° C. | Glycerol | |
| OPN-001-B | Liquid | 150 μMol in 50 mM HEPES | 0.5 μM or 0.05 μM |
| (holoenzyme) | Storage at | pH 8.0; 150 mM NaCl; 10% | |
| | −20° C. | Zn Acetate; 5% Glycerol | |

### Protocol

| Receptor | NMDA |
|---|---|
| Method | Patch clamp, whole cell, voltage clamp (−30 mV) |
| Dilution media | 140 mM NaCl, 3 KCl, 2.5 CaCl2, 10 Hepes, 10 glucose, |
| for OPN-001-A | 0.1 mM glycine, 5 μM strychnine, 300 nM TTX, 1 mM |
| and OPN-001-B | amiloride and 5 μM LaCl3 (pH 7.35) |
| Extracellular | 140 mM NaCl, 3 KCl, 2.5 CaCl2, 10 Hepes, 10 glucose, |
| medium | 0.1 mM glycine, 5 μM ZnCl2, 5 μM strychnine, 300 nM |
| for OPN-001-A | TTX, 1 mM amiloride et 5 μM LaCl3 (pH 7.35) |
| Extracellular | 140 mM NaCl, 3 KCl, 2.5 CaCl2, 10 Hepes, 10 glucose, |
| medium for | 0.1 mM glycine, 10 mM tricine, 5 μM strychnine, 300 nM |
| OPN-001-B | TTX, 1 mM amiloride and 5 μM LaCl3 (pH 7.35) |
| Intracellular | 100 mM CsCl, 30 mM CsF, 8 NaCl, 2.4 CaCl2, 5 EGTA, |
| medium | 10 Hepes, 4 Mg-ATP, 0.4 Na2-GTP (pH 7.35) |
| Protocol | Compounds OPN-001-A and OPN-B-001 were diluted in |
| | media without Tricine and without zinc. A solution of |
| | NMDA was applied at a concentration of 250 μM for |
| | which the current is maximal in cultured sensory neurons. |
| | After sealing and opening, the cell was held at a potential |
| | of −30 mV to remove the magnesium block of the NMDA |
| | current. After stabilization of the current (steady state |
| | stabilization) an injection of OPN-001 products, or zinc, |
| | was performed. |
| | Injection: 2000 μl of the product were injected directly |
| | into the recording chamber near the neuron. |
| Number of | 36 recorded neurons |
| cells recorded | |

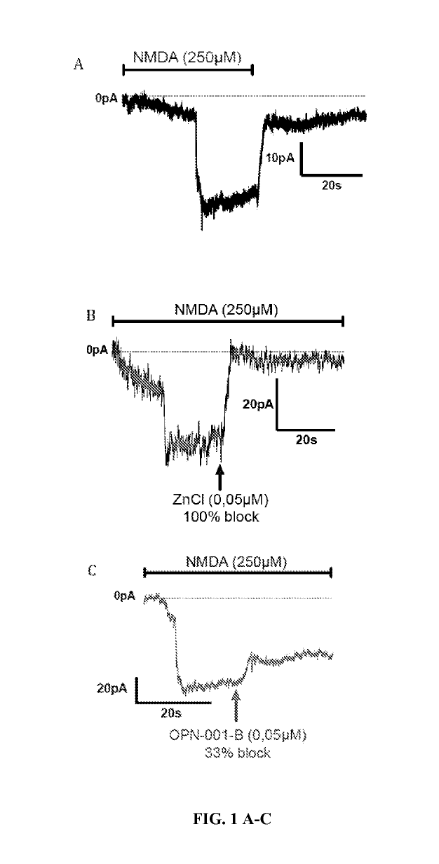

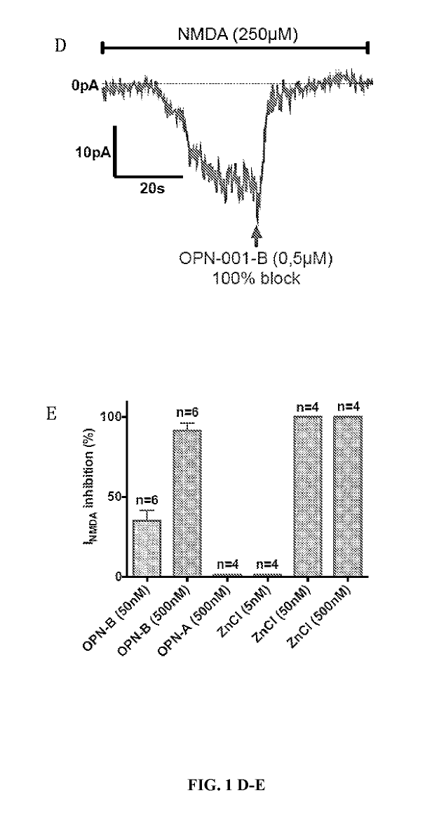

Electrophysiological traces are shown in FIG. 1.

OPN-001-B applied alone at a concentration of 500 nM, is not toxic (0/10 neurons) and causes a transient outward current of reduced amplitude.

As shown in FIGS. 1C, D and E, the application of OPN-001-B, at a concentration of 50 nM and 500 nM, inhibits 35% (16%-57%) and 92% (100%-77%) respectively of the induced current through the application of NMDA. The blocking effect occurs rapidly within 2 s for both concentrations.

As shown in FIGS. 1B and E, the application of ZnCl₂ at a concentration of 50 nM and 500 nM, inhibits 100% of the current induced by application of NMDA. The blocking effect also occurs within seconds. However, ZnCl₂ is not efficient alone at a concentration of 5 nM, which is the theoretical concentration of free Zn²⁺ in the OPN-001-B solution at 50 nM. Thus, the 35% NMDA current inhibition observed during the experiment can solely be related to the activity and presence of OPN-001-B.

These results thus confirmed the NMDA inhibitor activity of the enzyme of the invention.

1-18. (canceled)

19. An enzyme having a NMDA antagonist activity, wherein said enzyme is a metalloenzyme comprising at least one Zn²⁺ cation.

20. The enzyme according to claim 19, wherein said enzyme is selected from the group consisting of phosphotriesterases and phosphotriesterases derivatives.

21. The enzyme according to claim 19, wherein said enzyme is OPD or an OPD derivative.

22. The enzyme according to claim 19, wherein said enzyme is SEQ ID NO: 1, SEQ ID NO: 2, SEQ ID NO: 3, SEQ ID NO: 8 or SEQ ID NO: 53.

23. The enzyme according to claim 19, wherein said enzyme has an anticholinergic activity, a phosphotriesterase activity or both an anticholinergic activity and a phosphotriesterase activity.

24. The enzyme according to claim 19, wherein said enzyme is a phosphotriesterase derivative and is not capable of hydrolyzing an organophosphorous molecule selected from the group consisting of phosmet and fenthion.

25. A method for treating a NMDA related condition in a subject in need thereof, wherein said method comprises administering to the subject an enzyme having a NMDA antagonist activity, wherein said enzyme is a metalloenzyme comprising at least one divalent cation.

26. The method according to claim 25, wherein said at least one divalent cation is selected from the list comprising Zn2+, Mg2+, Ni2+, Cd2+, Mn2+, Co2+, Fe2+, and Ag2+.

27. The method according to claim 25, wherein said at least one divalent cation is Zn2+.

28. The method according to claim 25, wherein said enzyme is selected from the group consisting of phosphotriesterases and phosphotriesterases derivatives.

29. The method according to claim 25, wherein said enzyme is OPD or an OPD derivative.

30. The method according to claim 25, wherein said enzyme is SEQ ID NO: 1, SEQ ID NO: 2, SEQ ID NO: 3, SEQ ID NO: 8 or SEQ ID NO: 53.

31. The method according to claim 25, wherein the NMDA related condition is pain.

32. The method according to claim 25, wherein the NMDA related condition is hyperalgesia.

33. The method according to claim 25, wherein the NMDA related condition is opioid-induced hyperalgesia, hyperalgesia induced by other analgesics, or hyperalgesia induced by a chemotherapeutic agent or any other drug.

34. The method according to claim 25, wherein the NMDA related condition is neuropathy-associated pain or chronic brain impairment.

35. The method according to claim 25, wherein the NMDA related condition is neuropathy-associated pain selected from pain associated with neuropathy induced by a chemotherapeutic treatment, drug-induced neuropathy, or psychiatric medication induced neuropathy.

36. The method according to claim 25, wherein the NMDA related condition is excitotoxicity and/or is malfunctioning of glutamatergic neurotransmission.

37. The method according to claim 25, wherein the NMDA related condition is glutamate excitotoxicity.

38. A medical device coated with an enzyme having a NMDA antagonist activity, wherein said enzyme is a metalloenzyme comprising at least one Zn²⁺ cation.
